(19) United States
(12) Patent Application Publication
(10) Pub. No.: US 2008/0234267 A1
(43) Pub. Date: Sep. 25, 2008

(54) Compounds and Methods of Treatment

(76) Inventor: Karen Elizabeth LACKEY, Durham, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/688,298

(22) Filed: Mar. 20, 2007

(51) Int. Cl. A61K 31/4353; A61K 31/535

(52) U.S. Cl. 514/235.2; 514/352

(57) ABSTRACT

A derivative, which is useful as a ret kinase inhibitor is described herein. The described invention also includes methods of using the same in the treatment of diseases mediated by inappropriate ret kinase activity.

### FIELD OF THE INVENTION

The present invention relates to compounds, compositions and medicaments containing the same, as well as processes for the preparation and use of such compounds, compositions and medicaments. Such compounds are of potential therapeutic benefit in the treatment of diseases and conditions associated with inappropriate ret kinase activity, in particular in the treatment of cancer.

### BACKGROUND TO THE INVENTION

An important large family of enzymes is the protein kinase enzyme family. Currently, there are about 500 different known protein kinases. Protein kinases serve to catalyze the phosphorylation of an amino acid side chain in various proteins by the transfer of the y-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Accordingly, the protein kinase family of enzymes is typically classified into two main subfamilies: Protein Tyrosine Kinases (PTK) and Protein Serine/Threonine Kinases (PSTK), based on the amino acid residue they phosphorylate.

Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied family of enzymes in biochemical and medical research.

The ret (REarranged during Transfection) kinase is a receptor protein tyrosine kinase. Ret kinase is expressed in the thymus, salivary gland, spleen, lymph node, testes, and elements of the central and peripheral nervous system (Golden, J P et al., Experimental Neurology, 1999, 158, 504-528). Ret kinase is also expressed in tumors derived from neuroblastomas, pheochromocytomas, and medullary thyroid carcinoma (MTC) (Salomon, R. et al., Advances in Nephrology From the Necker Hospital, 1998, 28, 401-417). Ret kinase knock out mice exhibit severe developmental kidney anomalies and die within 24 hours after birth due to dysfunctional respiratory control secondary to cervical ganglia development anomalies. These mice also lack enteric neurons and have other nervous system anomalies suggesting that a functional ret kinase protein product is required during development (Taraviras, S. et al., Development, 1999, 126:2785-2797).

Aberrant ret kinase expression is associated with multiple endocrine neoplasia (MEN 2A and 2B), familial medullary thyroid carcinoma (FMTC), papillary thyroid carcinoma (PTC) and Hirschsprung's disease (HSCR). MEN 2A is a cancer syndrome resulting from a mutation in the extracellular cysteine-rich domain leading to dimerization via a disulfide bond which causes constituitive activation of the tyrosine kinase activity. Individuals with this mutation may develop medullary thyroid carcinoma (MTC), parathyroid hyperplasia, and pheochromocytoma. MEN 2B is caused by a MET⁹¹⁸→Thr mutation which changes the tyrosine kinase specificity. MEN 2B is similar to MEN 2A, but lacks the parathyroid hyperplasia and also leads to development of numerous mucosal ganglia of the lips, tongue, and intestinal tract. Chromosomal rearrangements linking the promotor and NH2-terminal domains or unrelated gene(s) to the COOH-terminus of ret kinase resulting in constituitively activated chimeric forms of the receptor (ret/PTC) are thought to be tumor initiating events in PTC. (Viglietto, G. et al., Oncogene, 1995, 11:1207-1210). PTC's encompass about 80% of all thyroid carcinomas.

Inhibition of ret kinase represents a specific therapeutic approach. Treatment with Indolinone RPI-1 inhibited cell proliferaton and induced G2 cell cycle accumulation via abolishment of ret/PTC1 tyrosine phosphorylation (Lanzi, C et al., Cellular and Molecular Life Sciences, 2003, 60: 1449-1459). Further, these studies demonstrated abolishment of JNK2 and AKT activation, consistent with downstream signal pathway elements. A pyrazolopyrimidine, PP2, has also been reported to inhibit the growth of two human papillary thyroid carcinoma cell lines that carry the ret/PTC1 rearrangements due to its potent inhibition of ret kinase enzymatic activity in the nanomolar range (Carlomagno, F. et al., J of Clin Endocrinology and Metabolism, 2003, 88:1897-1902). ZD6474, an orally active inhibitor of VEGFR2 activity, is reported to efficiently block oncogenic ret kinases (Carlomagno, F et al., Cancer Research, 2002, 62:7284-7290). Ret/PTC3 transformed cells treated with ZD6474 lost proliferative autonomy, showed morphological reversion, blocked anchorage-independent growth and blocked the formation of tumors after injection of NIH-ret/PTC3 cells into nude mice. Finally, another example of ret kinase mediation was demonstrated with four arylidene-2-indolinone compounds that inhibited ret/ptc1 activity in immunokinase assay with IC50 values in the 27-42 micromolar range (Lanzi, C. et al., International Journal of Cancer, 2000, 85:384-390). Following exposure to (1,3-dihydro-5,6-dimethoxy-3-[(4-hydroxyphenyl)methylene]-2H-indol-2-one, the transformed phenotype of NIH3T3ptc1 cells were reverted, within 24 hours, to a normal fibroblast-like morphology in adherent cell sulture. Data presented provide evidence that ret/PTC1 is implicated in malignant transformation, and demonstrate the ability of (1,3-dihydro-5,6-dimethoxy-3-[(4-hydroxyphenyl)methylene]-2H-indol-2-one to interfere in the signal transduction pathway constituitively activated by ret/ptc1 oncoprotein.

WO 02/068394 discloses the compound N,N-(2-methyl-5-hydroxyphenyl)-(6-methanesulfonylquinolin-4-yl)amine which exhibits activity at P56lck. P56lck is indicated in disease conditions in which T cells are hyperactive eg. Transplant rejection rheumatoid arthritis, SLE, graft vs. host disease, T-cell mediated hypersensitivity, multiple sclerosis, IBD psoriasis, Hashimoto's thyroiditis, Guillain-Barre syndrome, cancer, dermatitis, allergic disorders, asthma, ischemic or reperfusion injury, allergic rhinitis, burns, myocardial infarction, stroke, osteoarthritis, ulcerative colitis, Sjoegren's syndrome, autoimmune hyperthyroidism, Grave's disease, Addison's disease, alopecia, anemia, autoimmune hypopituatarism, glomerulonephritis, urticaria, scleracierma, vasculitis, insulin-dependent diabetes and ARDS. The process of angiogenesis has been associated with a number of disease states (eg. tumourogenesis, psoriasis, rheumatoid arthritis) and this has been shown to be controlled through the action of a number of receptor tyrosine kinases (L. K. Shawver, DDT, 1997 2(2), 50-63).

The present inventors have discovered that substituted 4-(3-hydroxyanilino)-quinoline compounds are a potent inhibitors of ret kinase. Substituted 4-(3-hydroxyanilino)-quinoline compounds thus are potentially useful in the treatment of disorders associated with inappropriate ret kinase activity, in particular thyroid cancer, more particularly, multiple endocrine neoplasia (MEN 2A and 2B) familial medullary thyroid carcinoma (FMTC), papillary thyroid carcinoma (PTC) and Hirschsprungs disease (HSCR).

### BRIEF SUMMARY OF THE INVENTION

In one aspect of the present invention, there is provided a compound of formula (I)

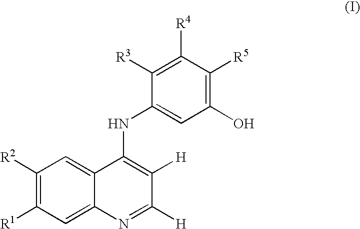

or a salt, or solvate thereof, wherein:

R¹ represents H, phenyl, a 5 or 6 membered monocyclic heteroaryl group, a bicyclic heteroaryl group, each of which phenyl, monocyclic or bicyclic heteroaryl group is optionally substituted by one or more substituents independently selected from
—C₁₋₃alkyl, —C₁₋₃hydroxyalkyl, —C₁₋₃haloalkyl, —C₁₋₃alkoxy, —C₁₋₃ haloalkoxy, —COH,
R^{a} and R^{b} are independently H, —C₁₋₃alkyl, —C₁₋₃alkyleneNR^{c}R^{d} or R^{a} and R^{b}, together with the N to which they are joined, form a 5 or 6 membered heterocyclic ring optionally containing a further heteroatom selected from 0 or N and optionally substituted by —C₁₋₃alkyl.
R^{c} and R_{d} are independently H, —C₁₋₃alkyl or
R^{c} and R^{d} together with the N to which they are joined form a 5 or 6 membered heterocyclic ring optionally containing a further heteroatom selected from O or N and optionally substituted by —C₁₋₃alkyl.

R² represents H, —SO₂C₁₋₃alkyl, —CONHC₁₋₃alkyl, a 6 membered monocyclic heteroaryl group.

R³, R⁴ and R⁵ independently represent H, halogen, —C₁₋₃alkyl with the proviso R¹ and R² are not both H and with the proviso that the compound is not N,N-(2-methyl-5-hydroxyphenyl)-(6-methanesulfonylquinolin-4-yl)amine:

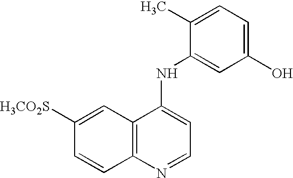

In a further aspect of the present invention, there is provided a pharmaceutical composition comprising a compound of formula (I), or a salt or solvate thereof and one or more of pharmaceutically acceptable carriers, diluents and excipients.

In a further aspect of the present invention, there is provided a compound of formula (I), or a salt or solvate thereof for use in therapy.

In a further aspect of the present invention, there is provided a compound of formula (I) or a salt or solvate thereof for use in the treatment of a disease or condition associated with inappropriate ret kinase activity including thyroid cancer, in particular multiple endocrine neoplasia (MEN 2A and 2B), familial thyroid carcinoma (FMTC), papillary thyroid carcinoma (PTC) and Hirschsprungs Disease (HSCR).

In a further aspect of the present invention, there is provided a compound of formula (Ia)

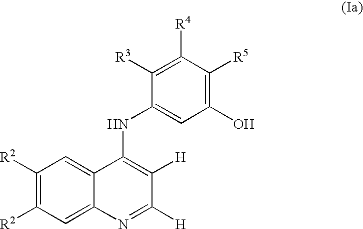

or a salt, or solvate thereof, wherein:

R¹ represents H, phenyl, a 5 or 6 membered monocyclic heteroaryl group, a bicyclic heteroaryl group, each of which phenyl, monocyclic or bicyclic heteroaryl group is optionally substituted by one or more substituents independently selected from
—C₁₋₃alkyl, —C₁₋₃hydroxyalkyl, —C₁₋₃haloalkyl, —C₁₋₃alkoxy, —C₁₋₃ haloalkoxy, —COH, —C₁₋₃alkyleneNR^{a}R^{b}
R^{a} and R^{b} are independently H, —C₁₋₃alkyl, —C₁₋₃alkyleneNR^{c}R^{d} or R^{a} and R^{b}, together with the N to which they are joined, form a 5 or 6 membered heterocyclic ring optionally containing a further heteroatom selected from O or N and optionally substituted by —C₁₋₃alkyl.
R^{c} and R^{d} are independently H, —C₁₋₃alkyl or
R^{c} and R^{d} together with the N to which they are joined form a 5 or 6 membered heterocyclic ring optionally containing a further heteroatom selected from O or N and optionally substituted by —C₁₋₃alkyl.
R² represents H, —SO₂C₁₋₃alkyl, —CONHC₁₋₃alkyl, a 6 membered monocyclic heteroaryl group.

R³, R⁴ and R⁵ independently represent H, halogen, —C₁₋₃alkyl with the proviso R¹ and R² are not both H, for use in the treatment of a disease or condition associated with inappropriate ret kinase activity including thyroid cancer, in particular multiple endocrine neoplasia (MEN 2A and 2B), familial thyroid carcinoma (FMTC), papillary thyroid carcinoma (PTC) and Hirschsprungs Disease (HSCR).

In a further aspect of the present invention, there is provided a method of treating a disease or condition associated with inappropriate ret kinase activity, including thyroid cancer, in particular multiple endocrine neoplasia (MEN 2A and 2B), familial thyroid carcinoma (FMTC), papillary thyroid carcinoma (PTC and Hirschsprungs Disease (HSCR) in a mammal comprising administering to said mammal a compound of formula (la) or a salt or solvate thereof.

In a further aspect of the present invention there is provided the use of a compound of formula (Ia) or a salt or solvate thereof in the manufacture of a medicament for use in the treatment of a disease or condition associated with inappropriate ret kinase activity including thyroid cancer, in particular multiple endocrine neoplasia (MEN 2A and 2B), familial thyroid carcinoma (FMTC), papillary thyroid carcinoma (PTC and Hirschsprungs Disease (HSCR).

### DETAILED DESCRIPTION OF THE INVENTION

As used herein, the term “effective amount” means that amount of a drug or pharmaceutical agent that will elicit the biological or medical response of a tissue, system, animal or human that is being sought, for instance, by a researcher or clinician.

Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms. As used herein, the term “C₁-C₃ alkyl” refer to an alkyl group, as defined above, containing at least 1, and at most 3 or 6 carbon atoms respectively. Examples of “alkyl” as used herein include, but are not limited to, methyl, ethyl, n-propyl, isopropyl, n-butyl, isobutyl, t-butyl, n-pentyl, isopentyl.

As used herein, the term “alkylene” refers to a straight or branched chain divalent hydrocarbon radical having the specified number of carbon atoms. As used herein, the terms “C₁-C₃ alkylene” refer to an alkylene group, as defined above, which contains at least 1, and at most 3 carbon atoms respectively. Examples of “alkylene” as used herein include, but are not limited to, methylene, ethylene, n-propylene, and the like.

As used herein, the term “halogen” refers to fluorine (F), chlorine (Cl), bromine (Br), or iodine (I) and the term “halo” refers to the halogen radicals: fluoro (—F), chloro (—Cl), bromo(—Br), and iodo(—I).

As used herein, the term “haloalkyl” refers to an alkyl group as defined above, substituted with at least one halo group, halo being as defined herein. Examples of such branched or straight chained haloalkyl groups useful in the present invention include, but are not limited to, methyl, ethyl, propyl, isopropyl, substituted independently with one or more halos, e.g., fluoro, chloro, bromo and iodo.

As used herein, the term “heterocyclic” or the term “heterocyclyl” refers to a non-aromatic heterocyclic ring, being saturated or having one or more degrees of unsaturation, containing one or more heteroatoms as defined and having the specified number of ring members.

As used herein, the term “alkoxy” refers to the group RₐO—, where Rₐ is alkyl as defined above and the terms “C₁-C₃ alkoxy” refer to an alkoxy group as defined herein wherein the alkyl moiety contains at least 1, and at most 3 carbon atoms. Exemplary “C₁-C₃ alkoxy” groups useful in the present invention include, but are not limited to, methoxy, ethoxy, n-propoxy.

As used herein, the term “haloalkoxy” refers to the group RₐO—, where R^{a} is haloalkyl as defined above and the term “C₁-C₃ haloalkoxy” refers to a haloalkoxy group as defined herein wherein the haloalkyl moiety contains at least 1, and at most 3, carbon atoms. Exemplary C₁-C₃ haloalkoxy groups useful in the present invention include, but are not limited to, trifluoromethoxy.

As used herein, the term “heteroaryl” refers to a monocyclic five or six membered aromatic ring, or to a bicyclic aromatic ring system comprising two fused such monoyclic five or six membered aromatic rings. These heteroaryl rings contain one or more nitrogen, sulfur, and/or oxygen heteroatoms, where N-oxides and sulfur oxides and dioxides are permissible heteroatom substitutions. Examples of “heteroaryl” groups used herein include furanyl, thiophenyl, pyrrolyl, imidazolyl, pyrazolyl, triazolyl, tetrazolyl, thiazolyl, oxazolyl, isoxazolyl, oxadiazolyl, oxo-pyridyl, thiadiazolyl, isothiazolyl, pyridyl, pyridazyl, pyrazinyl, pyrimidyl, quinolinyl, isoquinolinyl, benzofuranyl, benzothiophenyl, indolyl, indazolyl.

As used herein, the term “hydroxyalkyl” refers to an alkyl group as defined above substituted with at least one hydroxy, hydroxy being as defined herein. Examples of branched or straight chained “C₁-C₃ hydroxyalkyl” groups useful in the present invention include, but are not limited to, methyl, ethyl, propyl, isopropyl, substituted independently with one or more hydroxy groups.

As used herein, the term “optionally” means that the subsequently described event(s) may or may not occur, and includes both event(s), which occur, and events that do not occur.

As used herein, the term “substituted” refers to substitution with the named substituent or substituents, multiple degrees of substitution being allowed unless otherwise stated.

The term “ret inhibitor”, is used to mean a compound which inhibits ret kinase.

The term “ret mediated disease” or a “disorder or disease or condition mediated by inappropriate ret activity” is used to mean any disease state mediated or modulated by ret kinase mechanisms, in particular thyroid cancer, including multiple endocrine neoplasie (MEN 2A and 2B), familial medullary thyroid carcinoma (FMTC), papillary thyroid carcinoma (PTC) and Hirschsprung's disease (HSCR).

As used herein, “a compound of the invention” means a compound of formula (I) or (Ia) or a salt or solvate thereof.

As used herein, the term “solvate” refers to a complex of variable stoichiometry formed by a solute (in this invention, a compound of the invention or a salt thereof) and a solvent. Such solvents for the purpose of the invention may not interfere with the biological activity of the solute. Examples of suitable solvents include, but are not limited to, water, acetone, methanol, ethanol and acetic acid. Preferably the solvent used is a pharmaceutically acceptable solvent. Examples of suitable pharmaceutically acceptable solvents include water, ethanol and acetic acid. Most preferably the solvent is water.

The compounds of the invention may have the ability to crystallize in more than one form, a characteristic, which is known as polymorphism, and it is understood that such polymorphic forms (“polymorphs”) are within the scope of formula (I) and (Ia). Polymorphism generally can occur as a response to changes in temperature or pressure or both and can also result from variations in the crystallization process. Polymorphs can be distinguished by various physical characteristics known in the art such as x-ray diffraction patterns, solubility and melting point.

Particular compounds of formula (I) include:

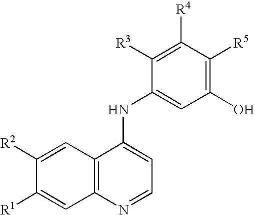

| Ex. | Chemical Name | R1 | R² | R³ | R⁴ | R⁵ |
|---|---|---|---|---|---|---|
| 1 | 3-{[7-(2-pyridinyl)-4-quino-linyl]amino}phenol | 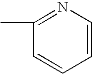 | H | H | H | H |
| 2 | 3-amino-5-{[7-(2-pyri-dinyl)-4-quino-linyl]amino}phenol | 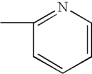 | H | H | CH₃ | H |
| 3 | 7-(1-benzofuran-2-yl)-4-quino-linyl]amino}-2-bromo-phenol | 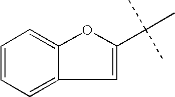 | H | H | H | Br |
| 4 | 2-chloro-4-fluoro-5-({7-[3-({[2-(4-morpho-linyl)ethyl]amino}meth-yl)phenyl]-4-quino-linyl}amino)phenol | 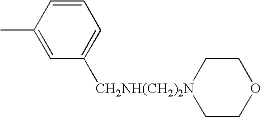 | H | F | H | Cl |
| 5 | 4-{(5-hydroxy-2-methyl-phenyl)amino]-7-quino-linyl}-1,3-thiazole-2-car-baldehyde | 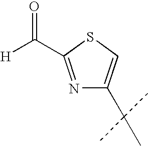 | H | CH₃ | H | H |
| 6 | 4-chloro-2-fluoro-5-{[6-(methy-sulfonuyl)-4-quino-linyl]amino}phenol | H | —SO₂CH₃ | Cl | H | F |
| 7 | 4-chloro-2-fluoro-5-({7-[2-(hy-droxymethyl)-1,3-thia-zol-4-yl]-4-quino-linyl}amino)phenol | 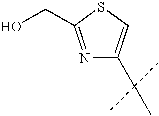 | H | Cl | H | F |
| 8 | 4-chloro-2-fluoro-5-({7-[2-(4-mor-pholinylmethyl)-1,3-thia-zol-4-yl]-4-quino-linyl}amino)phenol | 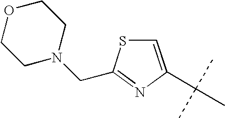 | —SO₂CH₃ | CH₃ | H | H |
| 9 | 4-methyl-3-{[6-(3pyri-dinyl)-4-quino-linyl]amino}phenol | H | 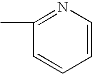 | CH₃ | H | H |
| 10 | 4-[(5-hydroxy-2-methyl-phenyl)amino]-N-(1-meth-ylethyl-6-quinoline-carboxamide | H | 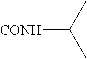 | CH₃ | H | H |

Particular compounds of formula (Ia) include:

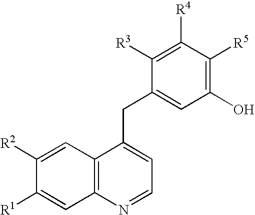

| Ex. | Chemical Name | R1 | R² | R³ | R⁴ | R⁵ |
|---|---|---|---|---|---|---|
| 1 | | 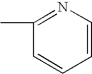 | H | H | H | H |
| 2 | | 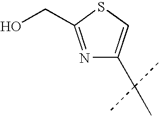 | H | H | CH₃ | H |
| 3 | | 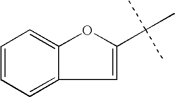 | H | H | H | Br |
| 4 | | 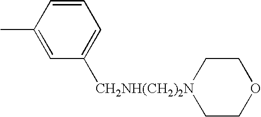 | H | F | H | Cl |
| 5 | | 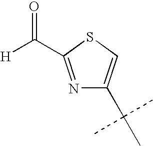 | H | CH₃ | H | H |
| 6 | | H | —SO₂CH₃ | Cl | H | F |
| 7 | | 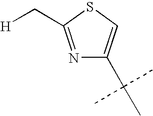 | H | Cl | H | F |
| 8 | | 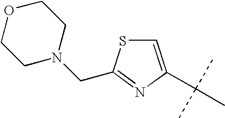 | —SO₂CH₃ | CH₃ | H | H |
| 9 | | H | 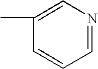 | CH₃ | H | H |
| 10 | | H | 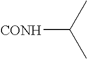 | CH₃ | H | H |
| 11 | | H | —SO₂CH₃ | CH₃ | H | H |

The compounds of the present invention may be in the form of and/or may be administered as a pharmaceutically acceptable salt. For a review on suitable salts see Berge et al, J. Pharm. Sci. 1977, 66, 1-19.

Typically, the salts of the present invention are pharmaceutically acceptable salts. Salts encompassed within the term “pharmaceutically acceptable salts” refer to non-toxic salts of the compounds of this invention.

Suitable pharmaceutically acceptable salts can include acid or base additions salts.

A pharmaceutically acceptable acid addition salt can be formed by reaction of a compound of with a suitable inorganic or organic acid (such as hydrobromic, hydrochloric, sulfuric, nitric, phosphoric, succinic, maleic, formic, acetic, propionic, fumaric, citric, tartaric, lactic, benzoic, salicylic, glutamaic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration. A pharmaceutically acceptable acid addition salt of a compound can comprise or be for example a hydrobromide, hydrochloride, sulfate, nitrate, phosphate, succinate, maleate, formarate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

A pharmaceutically acceptable base addition salt may, where there is a suitable acidic group, be formed by reaction of a compound with a suitable inorganic or organic base (e.g. triethylamine, ethanolamine, triethanolamine, choline, arginine, lysine or histidine), optionally in a suitable solvent such as an organic solvent, to give the base addition salt which is usually isolated for example by crystallisation and filtration.

Other suitable pharmaceutically acceptable salts include pharmaceutically acceptable metal salts, for example pharmaceutically acceptable alkali-metal or alkaline-earth-metal salts such as sodium, potassium, calcium or magnesium salts.

Other non-pharmaceutically acceptable salts, e.g. oxalates or trifluoroacetates, may be used, for example in the isolation of compounds of the invention, and are included within the scope of this invention.

The invention includes within its scope all possible stoichiometric and non-stoichiometric forms of the compounds of the invention.

The compounds of formula (I) and (Ia) and salts and solvates thereof, are believed to have therapeutic potential as a result of inhibition of the protein kinase ret.

The invention thus provides compounds of formula (I) and salts and solvates derivatives thereof for use in therapy, particularly in the treatment of diseases and conditions mediated by inappropriate ret kinase activity including thyroid cancer, in particular multiple endocrine neoplasia (MEN 2A and 2B), familial thyroid carcinoma (FMTC), papillary thyroid carcinoma (PTC) and Hirschsprungs Disease (HSCR).

The invention also provides compounds of formula (1a) for use in the treatment of diseases and conditions mediated by inappropriate ret kinase activity, including thyroid cancer, in particular multiple endocrine neoplasia (MEN 2A and 2B), familial thyroid carcinoma (FMTC), papillary thyroid carcinoma (PTC) and Hirschsprungs Disease (HSCR).

In a further aspect of the present invention, there is provided a method of treating a disease or condition associated with inappropriate ret kinase activity, including thyroid cancer, in particular multiple endocrine neoplasia (MEN 2A and 2B), familial thyroid carcinoma (FMTC), papillary thyroid carcinoma (PTC and Hirschsprungs Disease (HSCR) in a mammal comprising administering to said mammal a compound of formula (Ia) or a salt or solvate thereof.

In a further aspect of the present invention there is provided the use of a compound of formula (Ia) or a salt or solvate thereof in the manufacture of a medicament for use in the treatment of a disease or condition associated with inappropriate ret kinase activity including thyroid cancer, in particular multiple endocrine neoplasia (MEN 2A and 2B), familial thyroid carcinoma (FMTC), papillary thyroid carcinoma (PTC and Hirschsprungs Disease (HSCR).

The inappropriate ret activity referred to herein is any ret activity that deviates from the normal ret activity expected in a particular mammalian subject. Inappropriate ret activity may take the form of, for instances, an abnormal increase in activity, or an aberration in the timing and or control of ret activity. Such inappropriate activity may result then, for example, from over expression or mutation of the protein kinase leading to inappropriate or uncontrolled activation.

While it is possible that, for use in therapy, a compound the invention, as well as salts or solvates thereof, may be administered as the raw chemical, it is possible to present the active ingredient as a pharmaceutical composition. Accordingly, the invention further provides a pharmaceutical composition, which comprises a compound of formula (I) and salts or solvates thereof, and one or more pharmaceutically acceptable carriers, diluents, or excipients. The compounds of the formula (I) and salts or solvates thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical composition including admixing a compound of the formula (I), or salts, solvates and physiological functional derivatives thereof, with one or more pharmaceutically acceptable carriers, diluents or excipients.

Pharmaceutical compositions comprising compounds of the invention may be presented in unit dose forms containing a predetermined amount of active ingredient per unit dose. Such a unit may contain, for example, 5 μg to 1 g, preferably 1 mg to 700 mg, more preferably 5 mg to 100 mg of a compound of the invention depending on the condition being treated, the route of administration and the age, weight and condition of the patient. Such unit doses may therefore be administered more than once a day. Preferred unit dosage compositions are those containing a daily dose or sub-dose (for administration more than once a day), as herein above recited, or an appropriate fraction thereof, of an active ingredient. Furthermore, such pharmaceutical compositions may be prepared by any of the methods well known in the pharmacy art.

Pharmaceutical compositions may be adapted for administration by any appropriate route, for example by the oral (including buccal or sublingual), rectal, inhaled, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The formulation can also be prepared to prolong or sustain the release as for example by coating or embedding particulate material in polymers, wax or the like.

The compounds of the invention and salts and thereof, may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of the invention and salts and solvates thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories or as enemas.

Pharmaceutical compositions for nasal or inhaled administration wherein the carrier is a solid include a coarse powder having a particle size for example in the range 20 to 500 microns which is administered in the manner in which snuff is taken, ie. by rapid inhalation through the nasal passage from a container of the power held close up to the nose. Suitable compositions wherein the carrier is a liquid for administration as a nasal spray or as nasal drops, include aqueous or oil solutions of the active ingredient.

Pharmaceutical compositions adapted for administration by inhalation include fine particle dusts or mists, which may be generated by means of various types of metered, dose pressurised aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian. However, an effective amount of a compound of the invention for the treatment of diseases or conditions associated with inappropriate ret activity, will generally be in the range of 5 μg to 100 mg/kg body weight of recipient (mammal) per day and more usually in the range of 5 μg to 10 mg/kg body weight per day. This amount may be given in a single dose per day or more usually in a number (such as two, three, four, five or six) of sub-doses per day such that the total daily dose is the same. An effective amount of a salt or solvate, thereof, may be determined as a proportion of the effective amount of the compound of the invention per se.

The compounds of the present invention and their salts and solvates, thereof, may be employed alone or in combination with other therapeutic agents for the treatment of the above-mentioned diseases or conditions associated with inappropriate ret activity. In particular, combination with at least one other anti-cancer therapy is envisaged. In particular, in anti-cancer therapy, combination with other chemotherapeutic, hormonal or antibody agents is envisaged as well as combination with surgical therapy and radiotherapy. Combination therapies according to the present invention thus comprise the administration of at least one compound of the invention or a pharmaceutically acceptable salt or solvate thereof, and the use of at least one other cancer treatment method. Preferably, combination therapies according to the present invention comprise the administration of at least one compound of the invention or a pharmaceutically acceptable salt or solvate thereof, or a physiologically functional derivative thereof, and at least one other pharmaceutically active agent, preferably an anti-neoplastic agent. The compound(s) of the invention and the other pharmaceutically active agent(s) may be administered together or separately and, when administered separately this may occur simultaneously or sequentially in any order and by any convenient route. The amounts of the compound(s) of the invention and the other pharmaceutically active agent(s) and the relative timings of administration will be selected in order to achieve the desired combined therapeutic effect.

In one embodiment, an other anti-cancer therapy is at least one additional chemotherapeutic therapy. Such chemotherapeutic therapy may include one or more of the following categories of anti-cancer agents.

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristrine, vinblastine, vindesine and vinorelbine and taxoids like taxol and taxotere); and topoisomerase inhibitors (for example epipodophyllotoxins like etoposide and teniposide, amsacrine, topotecan and camptochecin);

(ii) cytostatic agents such as antioestrogens (for example tamoxifen, toremifine, raloxifine, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate) aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225], farnesyl transferase inhibitors, tyrosine kinase inhibitors and serine-threonine kinase inhibitors, for example inhibitors of the epidermal growth factor family (for example EGFR family tyrosine kinase inhibitors such as N-(3-chloro-4-fluorophenyl-7-methoxy-6-(3-morpholinoproproxy)quinazolin-4-amine (gefitinib, AZD 1839), N-3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinoproproxy)quinazolin-4-amine (CI-1033)), for example inhibitors of the platelet-derived growth factor family and for example inhibitors of the hepatocyte growth factor family;

(v) antiangiogenic agents such as those which inhibit the effects of vascular edothelial growth factor, (for example the anti-vascular endothelial cell growth factor antibody bevacizumab [Avastin™], and compounds that work by other mechanisms (for example linomide, inhibitors of integrin αvβ3 function and angiostatin);

(vi) vascular damaging agents such as Combretastatin A4;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(ix) immunotherapy approaches, including for example ex-vivo and in-vivo approaches to increase the immunogenecity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

When a compound of the invention is used in combination with a second therapeutic agent active against the same disease, the dose of each compound may differ from that when the compound is used alone. Appropriate doses will be readily appreciated by those skilled in the art.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Working Examples.

Compounds of general formula (I) and (Ia) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula (I). Those skilled in the art will recognize if a stereocenter exists in compounds of Formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

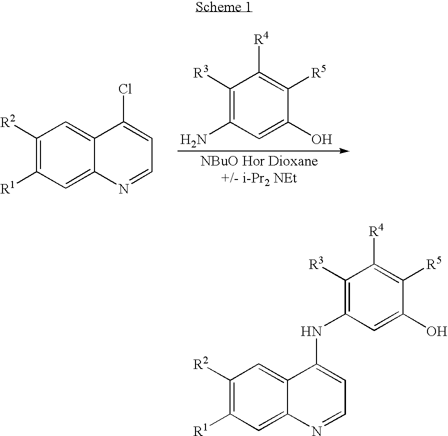

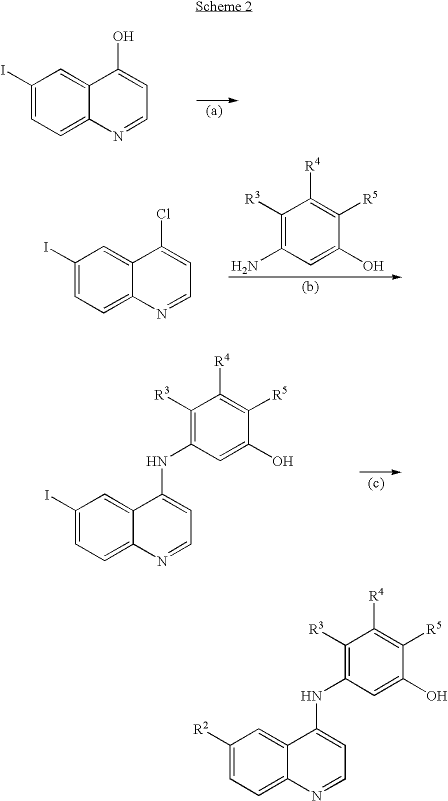

Steps b and c may be reversed eg. when R¹ is methylsulfonyl.

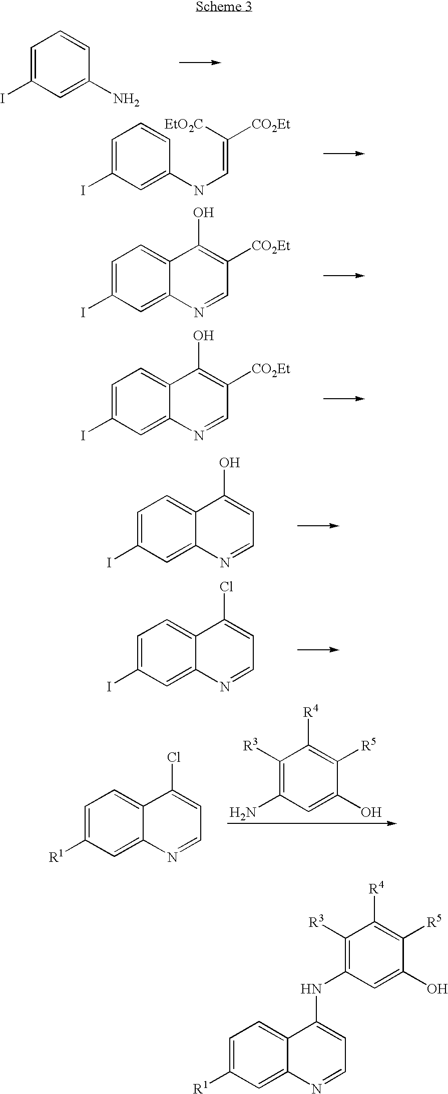

Certain embodiments of the present invention will now be illustrated by way of example only. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained or obtainable from commercial suppliers and used without further purification. Specifically, the following abbreviations may be used in the examples and throughout the specification:

| | |
|---|---|
| g (grams); | mg (milligrams); |
| l (litres); | ml (millilitres); |
| μl (microlitres); | M (molar); |
| MMol (millimolar); | mol (moles); |
| mmol (millimoles); | RT (room temperature); |
| min (minutes); | h (hours); |
| MeOH (methanol); | EtOAc (ethyl acetate); |
| DMF (N,N-dimethylformamide); | BuOH (buthanol); |
| EtOH (ethanol); | Me (methyl); |
| Et (ethyl); | |

### Example 1

### (GW440141A) 3-{[7-(2-pyridinyl)-4-quinolinyl]amino}phenol

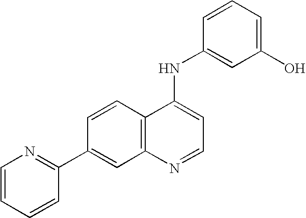

### Intermediate 1:

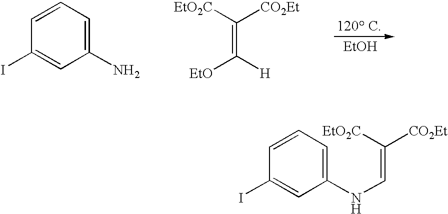

A mixture of 3-iodoaniline [84 g, 0.384 mol] and the malonate [120 ml] was heated at 120° C. for 1.5 hours. The ethanol produced during the reaction was removed in vacuo and the residue triturated in diethyl ether. A fluffy white solid was removed by filtration, washed with diethyl ether and dried at 60° C. in the vacuum oven. [12.427 g, 83%].

### Intermediate 2:

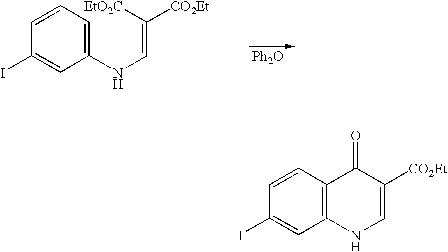

To a boiling diphenylether (260° C.) was added, portionwise, Intermediate 1 (20 g, 0.051 moles) over 5 mins at such a rate as to ensure the solid readily dissolved. After complete addition, the solution was heated for a further 15 mins before cooling to room temperature. Iso-hexane (400 ml) was added and the resultant precipitate was collected as a white solid by filtration. It was dried under vacuum overnight (approximately 18 hrs). (15.9 g, 91%).

### Intermediate 3:

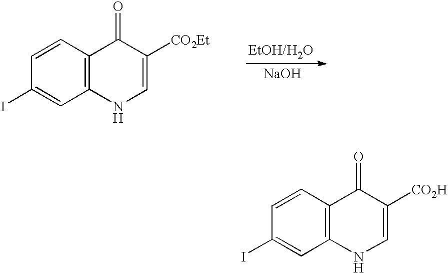

A suspension of Intermediate 2 (43.27 g, 126.1 mmol) in absolute ethanol (140 ml) and water (280 ml) was treated with sodium hydroxide pellets (43.27 g). The reaction mixture was heated to reflux for 45 minutes to afford a yellow solution and then allowed to cool to room temperature. Concentrated hydrochloric acid was added slowly with stirring and cooling in an ice-bath until the mixture was acidic. A while solid was removed by filtration, washed with water and dried in an oven (100° C.) overnight [36.83 g, 93%].

### Intermediate 4:

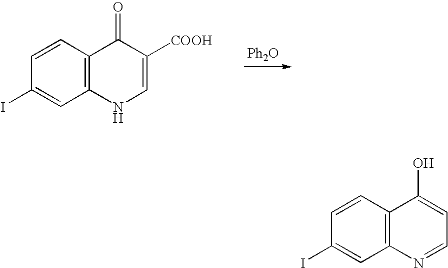

Intermediate 3 (0.125 mol) was added portionwise to boiling diphenyl ether, via an air condenser (violent reaction after each addition), over approximately 15 min. The resultant deep purple solution was heated for a further 5-10 min and then allowed to cool to room temperature with stirring. As the solution cooled the colour became lighter and a pale brown solid precipitated. The mixture was diluted with iso-hexane and the solid was collected by filtration. The solid was washed thoroughly with hexane to remove residual diphenyl ether and afforded desired product (32 g, 94%).

Intermediate 5: 4-chloro 7-iodocuinoline

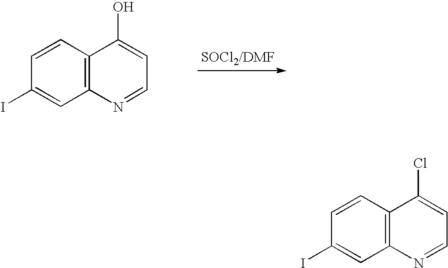

Thionyl chloride [130 ml] was added to Intermediate 4 (7.9 g, 29.15 mmol) to give a suspension. A few drops of dimethylformamide were added and the mixture was heated in an oil bath at 90° C. under nitrogen for approximately 18 hours. The solution was cooled and concentrated in vacuo. The resulting residue was azeotroped with toluene and the solid obtained was triturated using diethyl ether. A pale yellow solid was collected by filtration and washed with more ether to provide the desired product (9.003 g, 95%).

### Intermediate 6:

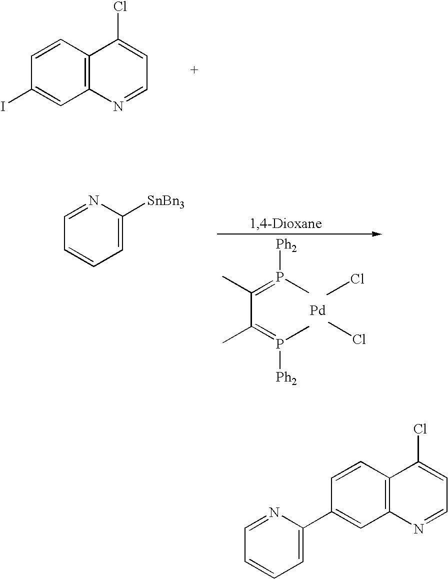

Intermediate 5 (1.589 g, 5.49 mmol), 2-(tributylstannyl) pyridine (2.42 g, 6.59 mmol), and palladium catalyst (1,4bis-(diphenylphosphine(Pd^{II}Cl2) (0.397 g) were heated under reflux in 1,4 dioxan (32 ml) under nitrogen. After 48 hr, the reaction was allowed to cool to room temperature and was then concentrated under vacume. Solvents (methanol and ethyl acetate) were added to the residue and the mixture was filtered to remove precipitate. The filtrate was concentrated in vacuo and chromatographed on silica gel using a gradient (10% ⁱHexane/EtOAc→EtOAc). Upon analytical examination of the isolated desired product, it was found to be contaminated with tin residues. Trituration using multiple solvents removed the tin residues, but product was also soluble in the solvents thus diminishing the yields. The product was finally dissolved in 2N HCl and the aqueous phase was extracted three times with diethyl ether. The aqueous layer was then basified with sodium bicarbonate and the resultant precipitate was collected by filtration (0.6 g, 45%). Some recovery of product from the aqueous layer was accomplished by extracting the basic aqueous filtrate with chloroform.

### Example 1

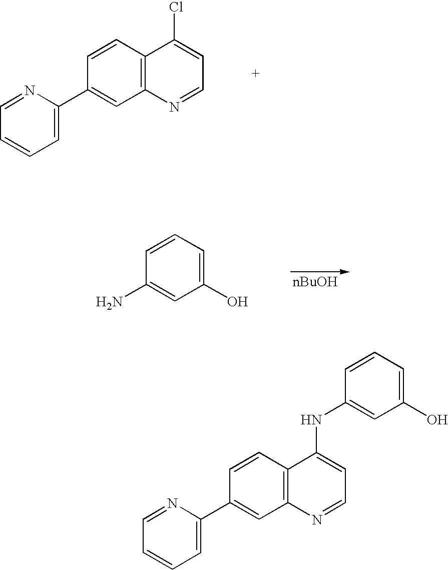

Intermediate 6 (50 mg, 0.2079 mmol) and an equivalent of the 3-hydroxyaniline (23 mg, 0.2079 mmol) were suspended in n-butanol (1 ml) and heated at 120° C. in a Stem Block apparatus for approximately 18 hours. The solvent had evaporated and the resulting residue was treated with fresh n-butanol. The solids were collected by filtration using an Alltechfrit and then washed with hexane to provide the desired product (49.4 mg, 68%).

### Example 2

### 3-amino-5-[{7-(2-pyridinyl)-4-quinolinyl]amino}phenol

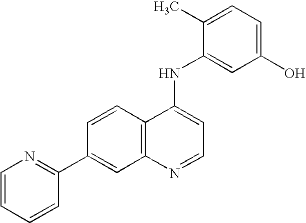

3-amino-5-{[7-(2-pyridinyl)-4-quinolinyl]amino}phenol was made in an analogous manner to the procedure to Example 1, by using 3-hydroxy-6-methylaniline (26 mg, 0.2079 mmol) in place of 3-hydroxyaniline to provide the desired product (50.8 mg, 67%).

### Example 3

### 5-[{7-(1-benzofuran-2-yl)-4-quinolinyl]amino}-2-bromophenol

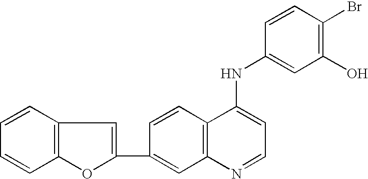

### Intermediate 7:

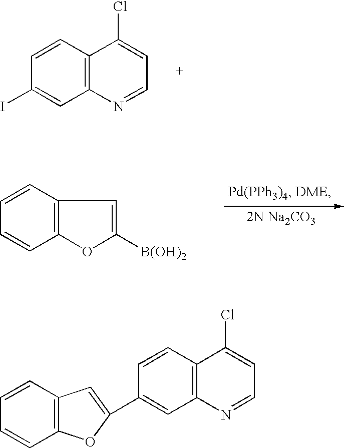

A mixture of Intermediate 5 (0.5 g, 1.73 mmol), benzo[b]furan-2-boronic acid (0.293 g, 1.92 mmol), tetrakistriphenylphosphine palladium catalyst (50 mg) and 2N sodium carbonate (7.78 ml) were heated under reflux in dimethoxyethane (15 ml) for approximately 18 hours. Fresh catalyst (50 mg) was added and heating continued for approximately an addition 72 hours. The solids from the reaction mixture were removed by filtration. The aqueous and organic layers were separated. The organic layer was washed with water and the aqueous washings were back extracted with EtOAc and combined with the organic phases. and al of the organic layers were combined. The combined organic layers were dried using a drying agent such as molecular sieves, filtered, and volatiles removed in vacuo. The resulting residue was chromatographed on silica gel using a gradient eluent of 2-6% EtOAc/ⁱHexane and the desired product was isolated after combining the appropriate fractions and removing solvent in vauo (18.6 mg, 38%).

### Example 3

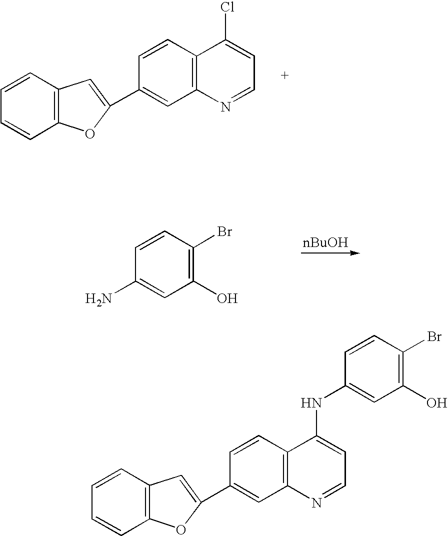

Intermediate 7 (50 mg, 0.179 mmol) and 3-hydroxy-4-bromoaniline (31 mg, 0.179 mmol) were suspended in 1 ml n-butanol and heated in a Stem Block apparatus at 120° for approximately 18 hours. The reaction mixture was cooled, diluted with hexanes, and solids were collected by filtration. The desired product was washed with hexanes and dried in vacuo (6 mg, 8%).

### Example 4

### 2-chloro-4-fluoro-5-({7-[3-({[2-(4-morpholinyl)ethyl]amino}methyl)phenyl]-4-quinolinyl}amino)phenol

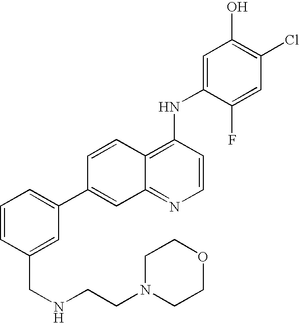

### Intermediate 8:

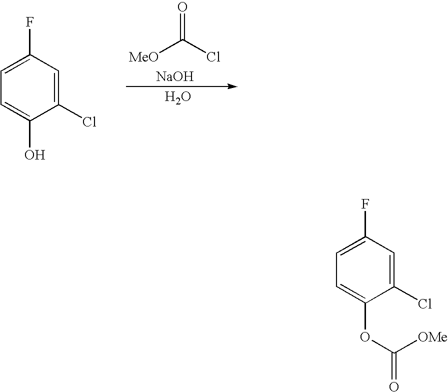

Sodium hychoride (10 g) and water (162 ml) were stirred in a 1 liter flask and cooled using an ice bath. 2-Chloro-4-fluoro-phenol (30 g, 0.0204 mol) was added. Methyl chloroformate (24.90 g, 20.4 ml) was added dropwise keeping the reaction temperature below 10° C. The reaction mixture was stirred for 10 minutes after complete addition before the solids were collected by filtration. The product was washed with water and dried to yield a white power (41.4 g, 98.9%).

### Intermediate 9:

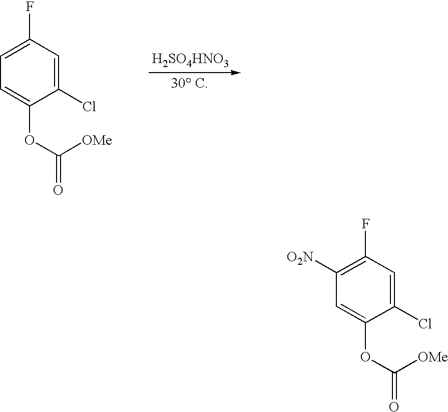

Intermediate 8 (65 g, 0.318 mol) was suspended in concentrated sulphuric acid (24.1 mls) and stirred in an ice bath. The nitrating mix of sulphuric acid (24.1 ml) and nitric acid (24.1 mls) was added slowly and turned the reaction yellow. Stirring continued until the reaction appeared to solidify. Water was added and the product was collected by filtration to yield the desired product with a small amount of water still present (81.47 g, 102%).

### Intermediate 10:

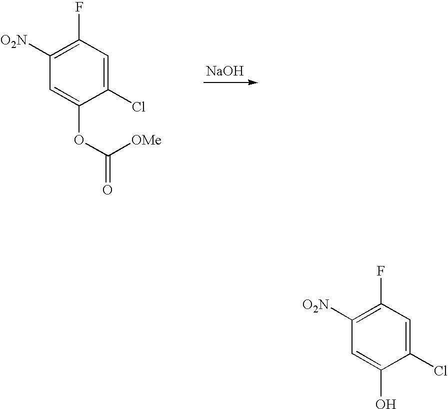

Intermediate 9 (93.64 g 0.375 mol), NaOH (17.7 g), H₂O (197 ml) were combined and refluxed for 4 hours. The reaction mixture was allowed to cool to room temperature and filtered through glass fiber filters. The filtrate was acidified with concentrated hydrochloric and the precipitate was collected by filtration,and dried in vacuo, to yield a brown powder (44.84 g, 62%).

### Intermediate 11:

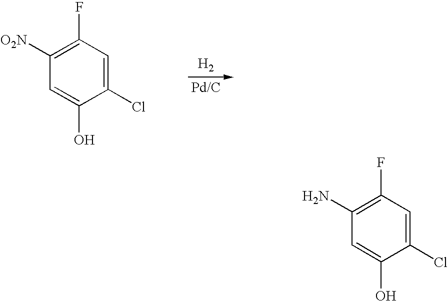

Pd/C (10%, 1 g) was charged to a flask under nitrogen followed by the addition of Intermediate 10 (10 g, 51.93 mmol) dissolved in EtOH (250 mL). The reaction was stirred for approximately 4 hours under H₂ (3739 ml, 155.8 mmol) and was then purged with nitrogen gas before removing the catalyst by filtration through a glass fiber pad. The filtrate was concentrated in vacuo to yield the desired product (8.08 g, 95.6%).

### Intermediate 12:

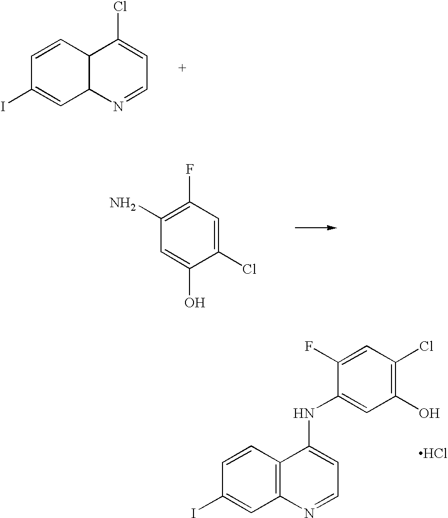

Intermediate 5 (10 g, 34.5 mmol) and Intermediate 12 (5.62 g, 34.5 mmol) were combined heated at 120° C. for 2 hours in 100 ml N-butanol. The reaction was cooled to room temperature and acetonitrile (300 ml) was added. The solids were collected by filtration and washed with fresh acetonitrile to yield a brown powder (13.35 g, 85,5%).

### Intermediate 13:

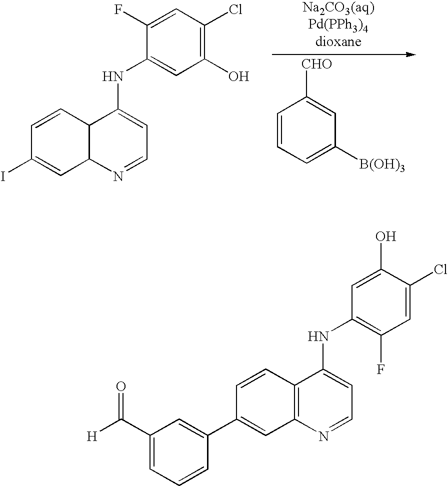

Intermediate 12 (2.0 g 4.81 mmol), dioxane (100 ml), 100 ml 2M Na₂CO₃(aq), 3-formyl phenyl boronic acid (865 mg, 5.77 mol), tetrakis-triphenylphosphine palladium catalyst (320 mg) were combined and stirred for approximately 16 hours at 80° C. The reaction mixture was cooled to room temperature and filtered through a glass fiber pad. The solvent was removed from the filtrate in vacuo. A biphasic mixture of ethylacetate and water was added to the resulting residue. An insoluble brown solid precipitated in both layers, which was collected by filtration and air dried using suction filtration to yield the desired product as brown powder (1.30 g, 68.6%).

### Example 4

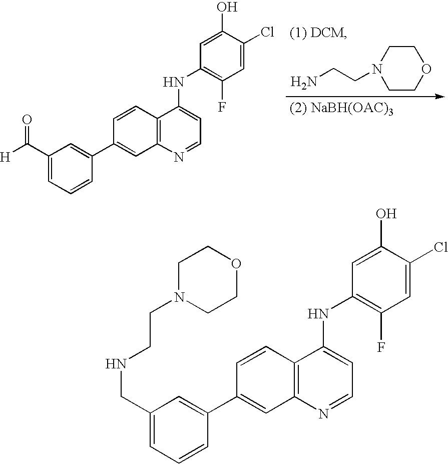

Dichloromethane (6 mL) was added to intermediate 13 (50 mg, 0.13 mmol) along with 2 large spatulas of molecular sieves. This reaction mixture was stirred for 5 minutes, then addition of N-aminoethylmorpholine (300 μl, excess) followed by acetic acid (1 mL) occurred. After stirring at room temperature, sodium triacetoxyborohydride (150 mg) was added. Once the reaction was complete, methanol was added to the reaction mixture and it was passed through an SCX cartridge, followed by elutions with MeOH and 10% NH₃/MeOH. The Ultraviolet active fractions were combined and solvent was removed in vacuo. The resulting residue was triturated, and the desired product (electrospray MS: 509.1 MH+) was collected by filtration.

### Example 5

### 4-{(5-hydroxy-2-methylphenyl)amino]-7-quinolinyl}-1,3-thiazole-2-carbaldehyde

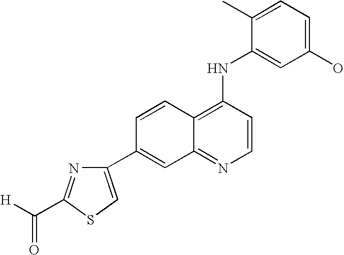

### Intermediate 14:

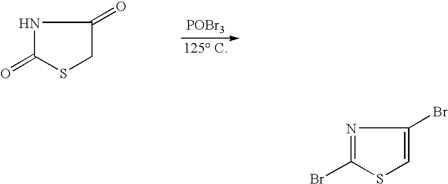

2-4-thiazolidinione (10 g, 0.085 mol) was added portionwise to stirred phosphorus oxybromide (160 g) at 80° C. The mixture was then warmed slowly to 125° C. with stirring (vigorous evolution of HBr occurred at approximately 118° C.) and held at 125° C. for 2 hrs. After cooling, the resultant solid was added portionwise to ice/sodium bicarbonate and the brown solid was collected by filtration. The reaction was repeated as above on 10 g and then on 20 g scale, and all three batches of the brown solids were combined. The crude combined material was purified via short path silica gel column chromatograph. The purified product was isolated as a pale yellow solid (41.23 g, 50%).

### Intermediate 15:

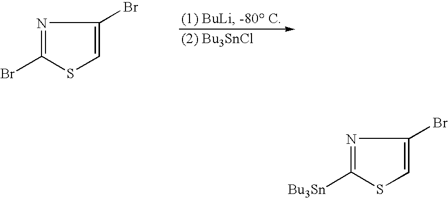

A solution of Bu₃SnCl (1.6M, 54 mL, 0.084 mol, 1.05 eq) was added dropwise via a dropping funnel to a cooled (−78° C.) solution of Intermediate 14 (20 g, 0.0823 moles) in dry ether (180 ml) under nitrogen. The resulting yellow-brown solution was stirred at −80° C. for 30 min before tributytin chloride (22.8 ml, 0.084 moles, 1.05 eg) was added using a dropping funnel at −80° C. The cooling bath was removed, allowing the reaction mixture to warm to −5° C. over 1 hr. The crude reaction was concentrated in vacuo followed by the addition of hexanes (200 ml), and the resultant precipitate was removed by filtration. The hexanes were removed from the filtrate in vacuo to provide the crude product (brown oil) which was purified by distillation (220° C., 0.56 mBar, Kugulrhor apparatus) in 2 batches, Batch 1 (8.81 g, 24%) and Batch 2 (23.07 g, 62%).

### Intermediate 16:

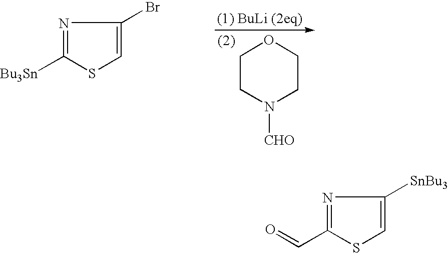

To a cooled solution (−90° C.), Intermediate 15 (8.81 g 0.019 moles) dissolved in THF (80 ml) under an inert atmosphere was added dropwise via a dropping funnel t-butyl lithium in pentane (28 mL, 1.5M, 2.2 eq, 0.042 mol) maintaining the internal reaction temperature below −85° C. The orange solution was stirred for a further 30 mins at −90° C. before N-formylmorpholine (4.2 ml, 0.041 mol, 2.1 eq) was added and stirred for a further 20 mins. The ice bath was removed and the reaction was allowed to warm to O° C. over 20 mins before the addition of water (100 ml) to the dark brown solution. The reaction mixture was extracted with ethyl acetate (3×100 ml) and the combined organic layers were washed with brine (50 ml), dried over MgSO₄ and conc in vacuo to yield the crude product as a brown oil (8 g). The reaction was repeated exactly as above, except the scale was doubled and the crude product was isolated as a brown oil (16 g). Both brown oils were combined and purified via flash silica gel column chromatography with a gradient elution (100% hexanes to 4/1 hexanes/diethyl ether. The purified product was collected in 2 fractions, fraction 1 (8.57 g, 39%) and fraction 2 (10.11 g, 47%)

### Intermediate 17:

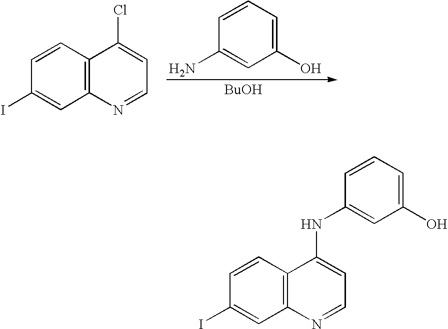

A suspension of Intermediate 5 (10 g 0.0345 moles) and 3-hydroxyaniline (4.25 g, 0.0345 moles) in BuOH (70 ml) was heated at reflux for 3.5 hrs. After cooling to room temperature, MeCN (200 ML) was added and the resultant precipitate was collected by filtration and washed with MeCN (2×100 ml). The desired product was dried and isolated as pale orange solid (12 g, 85%). The free base of the desired product was formed by heating the pale orange solid at 75° C. in chloroform (500 ml) and Na₂CO₃ (aq, 30 ml) for 45 mins. The product was collected by filtration and dried as a fine pale brown solid (8.20 g, 63%).

### Example 5

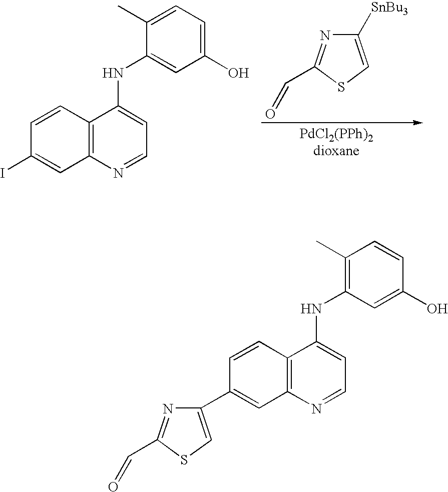

A suspension of Intermediate 17 (2 g, 5.32 mmol), Intermediate 16 (2.14 g, 5.32 mmol) and PdCl₂(PPh₃)₂ (0.747 g, 20 vol %) in dioxane (50 ml) was heated at reflux for approximately 18 hours. The cooled solution was filtered through a plug of celite to remove solids, and the filtrate was concentrated in vacuo. The resulting residue was triturated with hexanes (3×20 ml) to yield crude product, as an orange solid. The crude product was purified via flash column chromatography on silica gel, eluting with a gradient (5% EtOH/EtOAc to 20% EtOH/EtOAc). The appropriate fractions were combined, and solvent removed in vacuo to provide the desired product as a yellow solid (0.562 g, 29%).

### Example 6

### 4-chloro-2-fluoro-5-[{6-(methysulfonuyl)-4-quinolinyl]amino}phenol

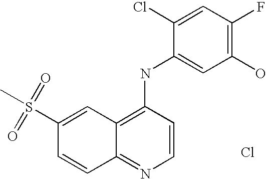

### Intermediate 18:

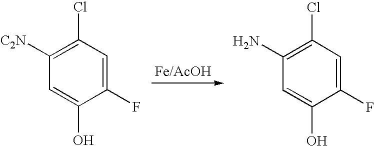

4-Chloro-2-fluoro-5-nitrophenol (15 g, 78.3 mmol), iron powder (11.745 g), acetic acid (150 mL) were combined and stirred for 3 hrs. Ethtyl acetate (400 mls) was added and the reaction mixture was neutralised with successive additions of solid Na₂CO₃. The ethyl acetate decanted off and the basic aqueous fraction was extracted with ethyl acetate (250 mL×3). The organic layers were combined, and extracted with H₂O (500 ml), saturated NaCl aq. (500 mL) and then dried over solid MgSO₄. The solids were removed by filtration and volatiles removed in-vacuo to yield the desired product as a solid (10.06 g, 79%).

### Intermediate 19:

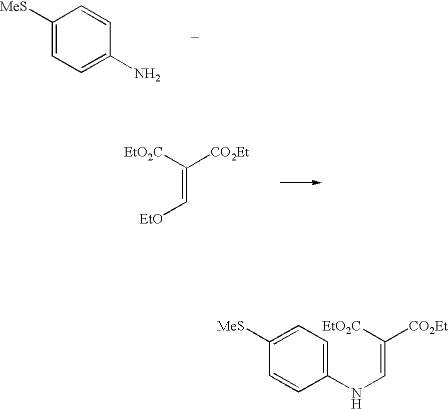

4-Methylmercaptoaniline (25 g, 0.18 mol) and diethylethoxymethylenemalonate (60 ml) were heated under reflux conditions for 2 hr. The condenser was removed after 1 hr to allow the ethanol produced to boil off. The mixture was allowed to cool to room temperature, and after approximately 72 hours, had solidified. The residue was treated with hexanes and the soleds were collected by filtration and dired to provide the desired product (45.8 g, 82.5%).

### Intermediate 20:

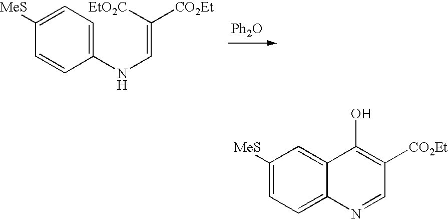

Intermediate 19 (49.7 g, 0.16 mol) was added portionwise to boiling diphenyl ether (300 ml). After complete addition, the reaction solution was heated for a further 15 min at reflux. The reaction was then allowed to cool to room temperature which resulted in the formation of solids. Hexanes were added to the mixture and the solids were collected by filtration. Hot hexanes were used to wash the solids to remove residual diphenyl ether and afford the purified desired product (36.9 g, 87.2%).

### Intermediate 21:

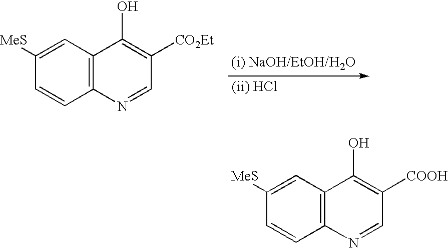

Intermediate 20 (36.8 g, 0.14 mcl), NaOH (47.1 g), water (250 ml) and ethanol (124 ml) were combined and the resulting suspension was heated under reflux for 45 minutes. The reaction mixture was allowed to cool to room temperature and concentrated HCl (103 ml) was added. The precipitate was collected by filtrationf, washed with water and dried under vacuum to provide the desired product. (31.4 g 95.9%).

### Intermediate 22:

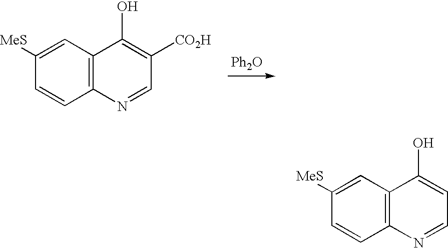

Intermediate 21 (31.4 g, 0.134 mol) was added portionwise to boiling diphenylether (500 ml) via an air condenser and heated under reflux for an additional 15 min. The reaction mixture was allowed to cool to room temperature and was then diluted with hexanes. The solids were collected by filtration, washed with hexanes, and dired to afford the desired product (25 g, 97.5%).

### Intermediate 23:

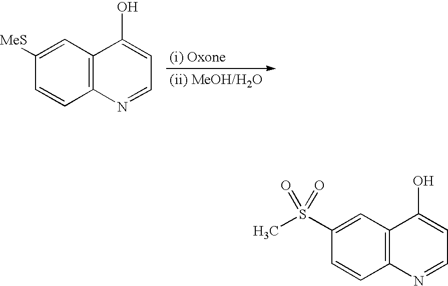

Intermediate 22 (1 g, 5.24 mmol) and oxone (3.5 g, 5.76 mmol) were suspended in a mixture of methanol (20 mL) and water (20 mL) and stirred at room temp continued for approximately 18 hours. The methanol was removed in vacuo. The precipitate in the remaining aqueous solution was removed by filtration and determined to be primarily inorganic material. Upon sitting, a precipitate formed in the aqueous filtrate, which was collected by filtration and washed with water, and set aside as crop 1. The resulting aqueous filtrate was concentrated in vacuo and the residue, crop 2, was combined with crop 1. The combined solids were treated with hot methanol, and inorganic solids were removed by filtration. The volatiles were removed from the filtrate to afford the desired product as a cream colored solid (0.81 g, 69%).

### Intermediate 24:

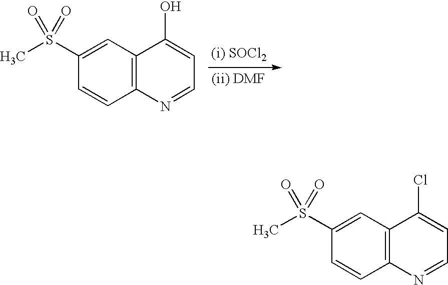

Intermediate 23 (0.5 g, 2.24 mmol) was combined with thionyl chloride (10 ml) and DMF (3 drops) was added. The reaction mixture was heated at reflux for 1 hour. The reaction was cooled and solvents were removed by azeotroping with toluene. The resulting residue was triturated with hexanes and the solid was collected by filtration and dried to afford the desired product (LC/MS 242 MH+).

### Example 6

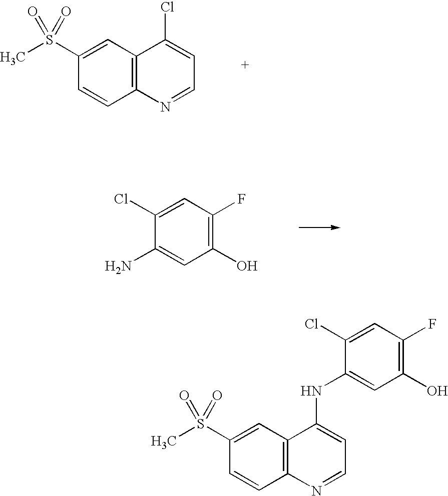

Intermediate 24 (50 mg 0.18 mmol) and Intermediate 18 (0.18 mmol) were combined and heated in a stem Block apparatus at 90° C. for approximately 18 hours. The reaction mixture was filtered while still hot and collected solids were washed with dioxane to provide the desired product.

### Example 7

### 4-chloro-2-fluoro-5-({7-[2-(hydroxymethyl)-1,3-thiazol-4-yl]-4-quinolinyl}amino)phenol

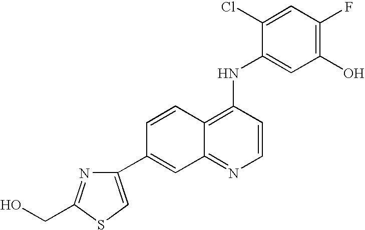

### Intermediate 25:

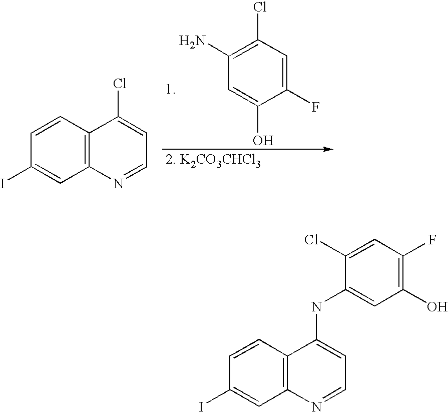

Intermediate 5 (1 g 3.46 mmol) and Intermediate 18 (0.562 g 3.4 mmol) were combined and heated in n-butanol (10 ml) at 120° C. for 17 h. Acetonitrile (100 ml) was added, and the solid was collected by filtration. The solids were washed with copious amounts of acetonitrile and air dried by suction filtration to yield the hydrochloride salt of the desired product (0.91 g, 58%). The free base was formed by combining the product with chloroform and saturated potassium carbonate and the mixture was stirred at 75° C. for 50 minutes. The reaction mixture was allowed to cool to room temperature and the solids were collected by filtration. The solids were washed with CHCl₃ and H₂O, and dried by suction filtration to yield a pale yellow powder.

### Intermediate 26:

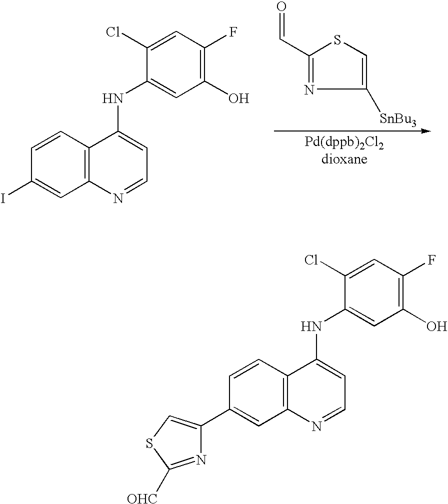

A suspension of Intermediate 25 (2 g, 4.82 mmol), Intermediate 16 (2.13 g, 5.3 mmol, 1.1 eq) and Pd(dppb)₂Cl₂ (0.49 g, 10 mol %) in dioxane (100 ml) was heated at reflux (120° C.) under an inert atmosphere for approximately 18 hrs. The crude reaction mixture was cooled to room temperature and filtered through a plug of celite. The filter cake was washed with EtOAc (150 ml), THF (100 ml) and chloroform (100 ml). The filtrate was concentrated in vacuo in the presence of silica gel. The resultant crude product on silica gel was purified via flash column chromatography on silica gel usind 5% EtOH in EtOAc as the eluent. The appropriate fractions were combined, and concentrated in vacuo to provide the purified product as an orange solid (0.87 g, 45%).

### Example 7

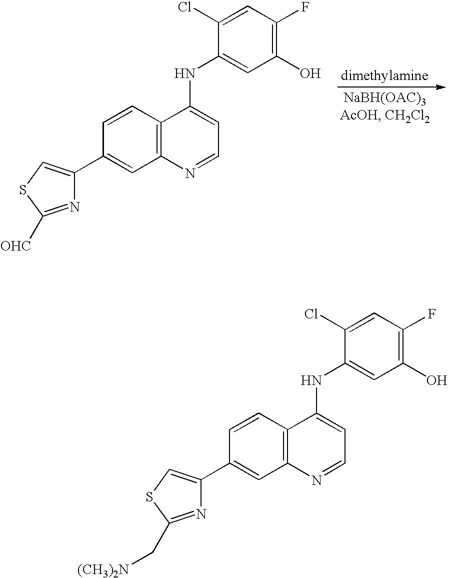

To a solution of Intermediate 26 (0.05 g) dissolved in dichloromethans DCM (6 ml) was added dimethylamine (0.3 ml) was stirred for 1 hour. 3A° molecular sieves were added and the reaction was stirred for a further 1 hour. Sodium triacetoxyborohydride (0.12 g) was added and the reaction was stirred at room temperature for approximately 18 hrs. Once the reaction was complete, methanol was added to the reaction mixture and it was passed through an SCX cartridge, followed by elutions with MeOH and 10% NH₃/MeOH. The Ultraviolet active fractions were combined and solvent was removed in vacuo. The resulting residue was triturated, and the desired product was collected by filtration and dried in vacuo (21 mg, 37%).

### Example 8

### 4-chloro-2-fluoro-5-({7-[2-(4-morpholinylmethyl)-1,3-thiazol-4-yl]-4-quinolinyl}amino)phenol

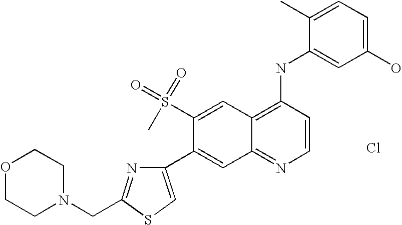

### Intermediate 27:

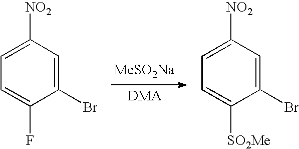

Methane sulfonic acid sodium salt (5.8 g, 56.9 mmol, 1.25 eq) was added to a stirred solution of 3-bromo-4-fluoronitrobenzene (10 g, 45.5 mmol) in N,N,-dimethylacetamide (50 ml). The mixture stirred at room temperature under nitrogen for approximately 18 hours. Water [200 ml] was added, that the resulting precipitate was collected by filtration, washed with water and air dried by suction filtration to afford the desired product as a white solid (12.0063 g 94%).

### Intermediate 28

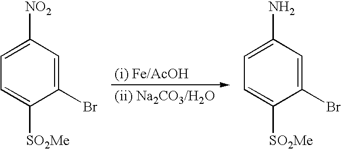

A suspension of Intermediate 27 [12 g, 42.86 mmol] in glacial acetic acid [150 ml] was cooled in an ice bath and iron powder [9.6 g, 171.43 mmol, 4 eq] was added portionwise with stirring. Heat evolved and the mixture became a very thick suspension. Stirring was continued at 0° C. for 20 minutes and at room temperature for a further 4 hours. The reaction mixture was poured slowly (over 20 minutes) into a biphasic mixture of water (100 mL), ethyl acetate [400 ml] and 2N sodium hydroxide aqueous solution [300 ml] with stirring and ice-cooling. Solid sodium carbonate was then added portion wise until effervescence ceased to reach a pH of 7. The layers were allowed to separate. The ethyl acetate layer was decanted off. More ethyl acetate (300 ml) was added to the aqueous layer with stirring and once settled decanted off. This was repeated once more. The combined ethyl acetate layers were dried over solid Na₂SO₄ and concentrated in vacuo to give the product as a pale yellow solid (9.41 g 88%].

### Intermediate 29:

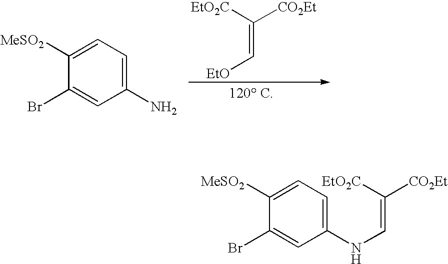

A mixture of Intermediate 28 [15.8 g, 63.2 mmol] and diethylethoxymethylene malonate [55 ml] was heated at for 1.25 hours at 120 ° C. The reaction was cooled to room temperature, and hexanes (150 mL) were added with vigorous stirring. A sticky white solid was removed by filtration, washed with hexanes and diethyl ether. The desired product was air dried by suction filtration dry to provide a fine, white powder [23.3 g, 88%].

### Intermediate 30:

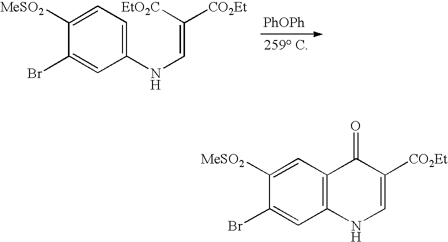

Diphenyl ether [200 ml] was heated to reflux (bp 259° C.) with stirring and Intermediate 29 [23 g, 54.76 mmol] was added portionwise over 20 minutes through an air condenser. Once the addition of solid was complete, the mixture was heated under reflux for a further 1.25 hours. The reaction mixture was cooled to room temperature and hexanes [400 ml] were added with stirring. A cream colored solid was collected by filtration and washed with hexanes, followed by diethyl ether and air dried to afford te desired product 18.88 g, 87%].

### Intermediate 31:

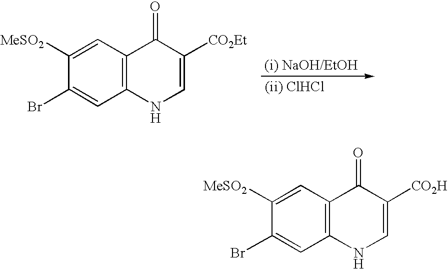

Intermediate 30 [17.8 g, 47.6 mmol] was suspended in ethanol [85 ml] and sodium hydroxide [17.8 g] in water [170 ml] was added. The mixture was heated under reflux for 1 hour. The reaction mixture was cooled using an ice-bath and concentrated hydrochloric acid [40 ml] was added with stirring. A peach colored solid was collected by filtration, washed with water, dry under vacuum filtration for approximately 72 hours. To remove the remaining water, the desired product was dried in the vacume oven at 80° C. for an hour [16.17 g, 98%].

### Intermediate 32:

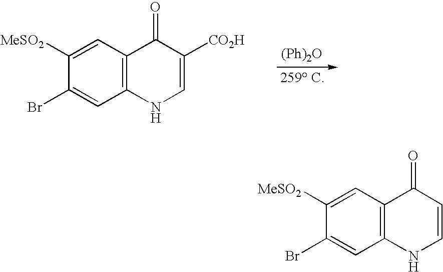

Intermediate 31 [6 g, 17.3 mmol] was added portionwise, over 20 minutes, through an air condenser to boiling diphenyl ether [170 ml, BP 259° C.] and the resulting suspension was heated under reflux for a further 3 hours. The charred looking mixture was cooled to ca. 40° C., diluted with hexanes and the solids collected by filtration. The brown solid was washed with hexanes and diethyl ether. The brown solid was heated in boiling methanol. A dark brown solid was removed by filtration and washed with 2 alliquots of hot methanol. The methanol filtrates were combined and concentrated in vacuo to give the desired product as a yellow solid [1.544 g, 30%].

### Intermediate 33:

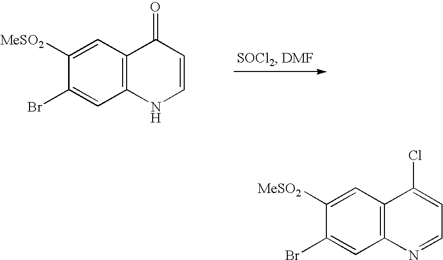

Intermediate 32 [1.5 g, 5 mmol] was suspended in thionyl chloride [25 ml], treated with a few drops of dimethylformamide and heated in an oil bath at 90° C. for 2 hours. The reaction misture was cooled to room temperature and concentrated in vacuo. The residue was azeotroped with toluene and then partitioned between 2M sodium hydroxide [100 ml] and dichloroethane [100 ml]. The layers were separated and the aqueous layer re-extracted with dichloromethane [100 ml]. The combined dichloromethane extracts were dried over Na₂SO₄, filtered to remove solids, and concentrated in vacuo to give the desired product as a yellow solid [1.532 g, 96%].

### Intermediate 34:

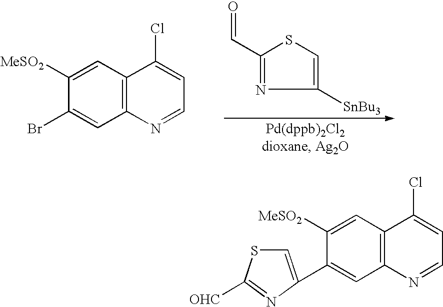

A mixture of Intermediate 33 [2.5 g, 7.8 mmol] and Intermediate 16 [3.47 g, 8.6 mmol] in dioxane [150 ml] was treated with i1,4 bis (diphenyl-phosphino)butane]palladium (ii) chloride [0.942 g, 1.56 mmol, 0.2 eq] and silver oxide [1.81 g, 7.8 mmol] and heated under reflux under nitrogen for 16 hours. The cooled reaction mixture was pre-absorbed onto silica gel (Merck 9385) and purified by flash column chromatography on silica eluting with a gradient of hexanes/ethyl acetate from 2:1 ratio to 1:2 ratio of solvents. The appropriate fractions were combined and concentrated in vacuo to give the product as a pale yellow solid [1.20 g, 44%].

### Intermediate 35:

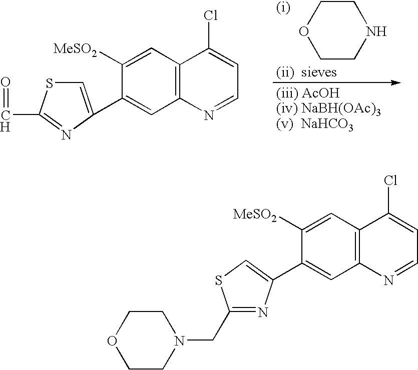

A solution of Intermediate 34 [0.28 g, 0.79 mmol] and morpholine [3.18 mmol, 4 eq] in dichloromethane [50 ml] was stirred at room temperature for 1 hour. Molecular sieves (4A) were added and stirring continued for 1 hour. Acetic acid [6 ml] was added and stirring continued for 1 hour. Sodium-triacetoxyborohydride [0.673 g, 3.18 mmol] was added and stirring continued for a further 18 hours. Sodium bicarbonate solution [2N] was added until effervescence had ceased. The layers were separated the the aqueous layer extracted with dichloromethane [25 mL]. The combined dichloromethane extracts were dried over MgSO₄, filtered to removed solids and the filtrate was concentrated in vacuo to produce crude product. Purification was accomplished by flash column chromagraphy on silica gel [Merck 9385] eluting with dichloromethane:ethanol:ammonia 200:8:1 The appropriate fractions were combined and solvents were removed in vacuo (LC/MS 425 MH+).

### Example 8

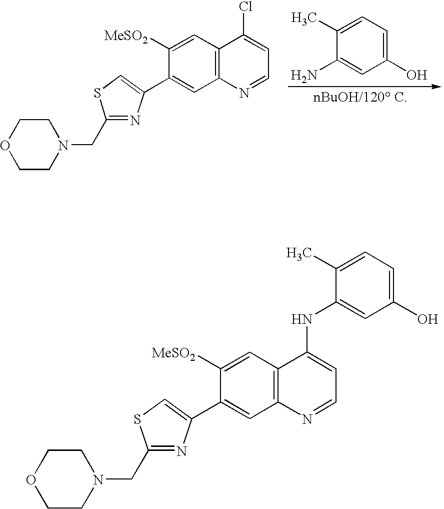

A mixture of Intermediate 35 [25 mg, 0.06 mmol] and 3-hydroxy-6-methylaniline [8 mg, 0.06 mmol] in n-butanol [42 ml] was heated at 120° C. under nitrogen for 19 hours. The n-butanol was removed in vacuo and the residue triturated using acetonitrile. The solids were collected by filtration, washed with acetonitrile and dried using suction filtration to afford the desired product as a dark yellow solid (19 mg, 59%).

### Example 9

### 4-methyl-3-{[6-(3pyridinyl)-4-quinolinyl]amino}phenol

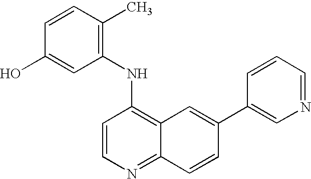

### Intermediate 36:

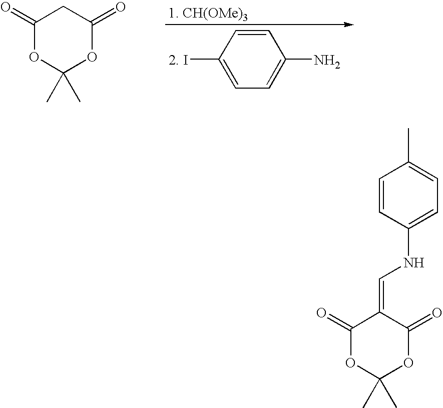

Meldrum's acid (7.5 g, 52.6 mmol) in CH(OMe)₃ (60 mL) was refluxed for 1 hour. 4-Iodoaniline (9.6 g 43.8 mmol) was added which immediately generated a precipitate and the reaction mixture was stirred for 1 hour at 110° C. This reaction was cooled to room temperature and the solids were collected by filtration. The solids were washed with MeOH and dried in vacuo to afford the desired product (10.5 g, 64%).

### Intermediate 37:

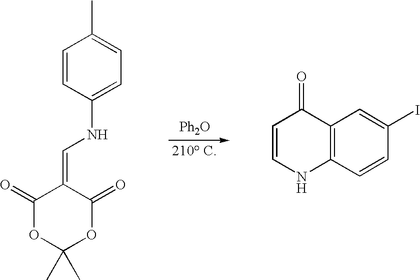

Intermediate 36 (9.5 g) was combined with diphenyl ether (90 mL) and the reaction vessel was lowered into a preheated oil bath (210° C.) and stirred for 8 minutes. The resulting clear brown solution was cooled to room temperature and hexanes (90 mL) were added. The precipative was collected by filtration and the desired product dried under reduced pressure (4.5 g, 65%).

### Intermediate 38:

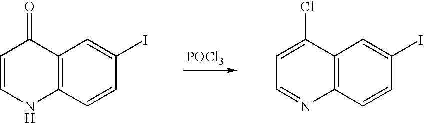

Intermediate 37 (4.9 g, 15.5 mmol) was combined with phosphorus oxychloride (14 mL, 15.5 mmol) and heated at 110° C. for 1 hour. The volatiles were removed in vacuo and the residue was treated with the biphasic mixture of saturated aqueous NaHCO3 and ethyl acetate. The layers were separated and the aqueous phase was extracted with ethyl acetate. The organic layers were combined, dried over solid Na₂SO₄, filtered and volatiles removed from the filtrate. The resulting residue was purified by silica gel column chromatography to afford the desired product (3.81 g, 85%).

### Intermediate 39:

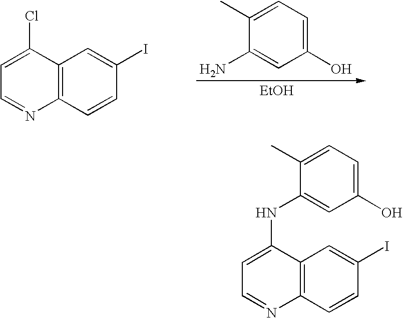

Intermediate 38 (1 g, 3.45 mmol), 3-hydroxy-6-methylaniline (0.51 g, 4.1 mmol) and ethanol (35 mL) were combined and was stirred at 70° C. for approximately 6 days.

Solvent was evaporated and the resulting residue was purified by silica gel using a gradient of CHCl₃/MeOH of 30/1 to 6/1. The appropriate fractions were combined, solvent removed in vacuo, and the desired product obtained (1.2 g, 93%).

### Example 9

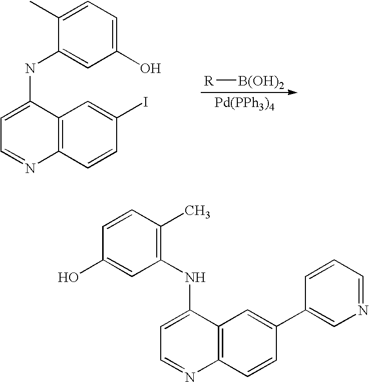

Intermediate 39 (40 mg, 0.11 mmol), tetrakistriphenylphosphine pallium catalyst (24.5 mg, 20 mol %), pyridylboronic acid (34.2 mg, 0.159 mmol), 2 M aqueous Na₂CO₃ (265 μL, 5 eq) and DMF (2 mL) were combined and stirred at 80° C. for approximately 18 hours (LC/MS 328 MH+).

### Example 10

### 4-[(5-hydroxy-2-methylphenyl)amino]-N-(1-methylethyl-6-quinolinecarboxamide

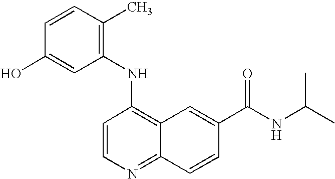

### Intermediate 40:

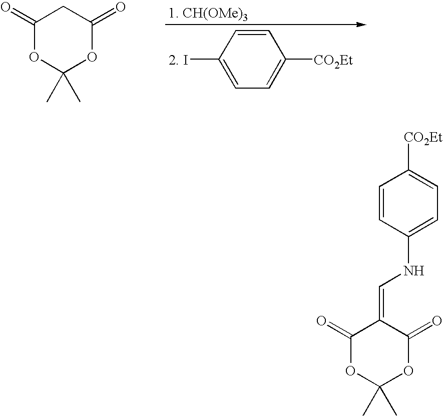

Meldrum's acid (7.5 g, 52.6 mmol) in CH(OMe)₃ (60 mL) was refluxed for 1 hour. 4-ethylcarboxyaniline (7.2 g 43.8 mmol) was added which immediately generated a precipitate and the reaction mixture was stirred for 6 hours at 110° C. This reaction was cooled to room temperature and the solids were collected by filtration. The solids were washed with MeOH and dried in vacuo to afford the desired product (9.8 g, 70%).

### Intermediate 41:

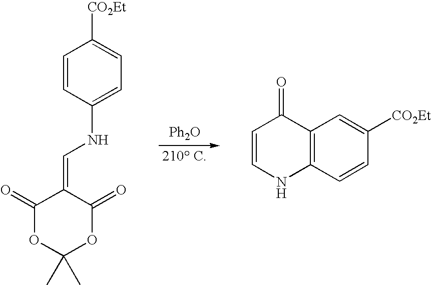

Intermediate 40 (9.8 g) was combined with diphenyl ether (70 mL) and the reaction vessel was lowered into a preheated oil bath (180° C.) which formed a suspension. The reaction was warmed 210° C. and this formed a solution which was stirred for between 10 and 15 minutes. The reaction was cooled to room temperature and hexanes (100 mL) were added. The precipative was collected by filtration and the desired product dried under reduced pressure (5.96 g, 89%).

### Intermediate 42:

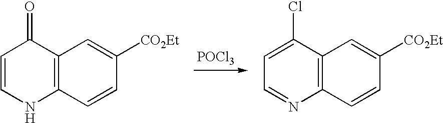

Intermediate 41 (3 g 13.8 mmol) and POCl₂ (8 mL) were combined and stirred for 3 h at 120° C. The solvent was evaporated in vacuo and crushed ice was added. A biphasic mixture of Saturated aqueous NaHCO₃ and ethyl acetate was added. The layers were separated and the aqueous phase was extracted with ethyl acetate. The organic phases were combined, dried over solid Na2SO4, filtered, and the volatiles from the filtrate were evaporated in vacuo. The desired product was purified by Silica gel column chromatography (2.19 g, 67%).

### Intermediate 43:

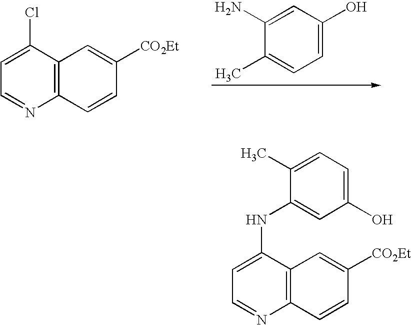

Intermediate 42 (1 g, 4.2 mmol), 3-hydroxy-6-methylaniline (0.62 g, 5 mmol) and isopropanol (30 mL) were combined and heated at 100° C. for 8 hours. The resulting suspension was cooled to room temperature and the solids were collected by filtration. The solids were washed with fresh isopropanol and dried in vacuo to give the desired product (0.77 g, 57%).

### Intermediate 44:

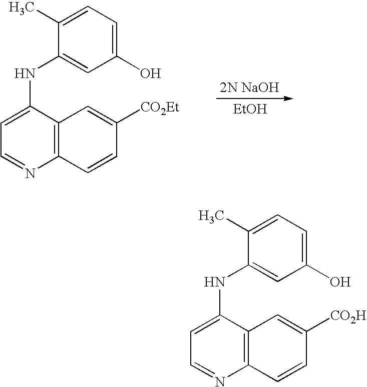

Intermediate 43 (0.70 g, 2.2 mmol) was suspended in ethanol (30 mL) and 2N NaOH (aq., 1.5 mL) was added and stirred at room temperature for 4 hours. Water (20 mL) was added and the reaction was stirred for another 4 hours. The ethanol was removed in vacuo, THF (20 mL) was added to the resulting aqueous solution and the reaction mixture was stirred for approximately 18 hours. An additional amount of 2N NaOH (aq., 5.5 mL) was added. The solvent was evaporated in vacuo and 2N HCl was added until the aqueous solution was acidic. A precipitate formed and was collected by filtration, and dried to provide the desired product (0.67 g, 92%)

### Example 10

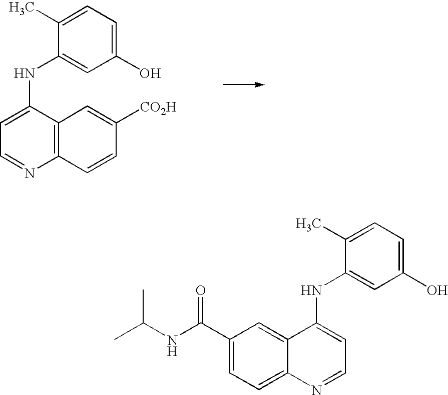

The following reagents were combined in order: isopropyl amine (7.1 mg, 0.12 mmol), Intermediate 44 (30 mg, 0.1 mmol), triethylamine (21 uL, 0.12 mmol), HOBt (18 mg, 0.12 mmol), WSC.HCl (23 mg, 0.12 mmol) and were dissolved in DMF (3 mL). The reaction was stirred at room temperature for 3 hours. Volatiles were removed in vacuo and the crude reaction misture was purified using an SCX column chromatography and eluting with 2N NH₃/CHCl₃: MeOH. The appropriate fractions were combined, volatiles removed and the desired product obtained.

### Example 11

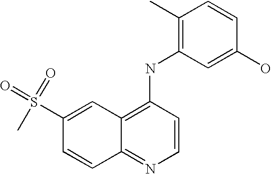

Preparation may be as described in WO 02/068394.

### Biological Data

The compound of the present invention was tested for ret kinase inhibitory activity in a ret kinase enzyme assay and in cell proliferation assays.

### Ret Kinase Enzyme Assay:

The ret kinase assay used the intracellular portion of ret (amino acids 713-1072 of the short isoform) produced in SF9 insect cells and purified on a nickel column. The assay was performed in a 45 μL volume with increasing concentrations of tyrosine kinase inhibitor (TKI) in triplicate in a 96-well plate format as follows:
4.5 μL 500 mM Morpholinopropanesulfonic acid, pH 7.4
4.5 μL 100 μM adenosine triphosphate
4.5 μL 250 mM MgCl₂
1.0 μL γ-ATP³³ (1 microCi)(3000 Ci/mMol)
2.0 μL (5 mg/ml) poly Glutamic acid-Tyrosine (Sigma (P-0275))
23 μL H₂O
1.0 μL recombinant ret (100 ng)
4.5 μL 10× Tyrosine kinase inhibitor

The components were incubated in triplicate for 30 minutes at room temperature within a 96-well plate in triplicate. The reaction was stopped by adding 45 μL 0.5% H₃PO₄ to each well. 75 μL of the reaction mixture was spotted onto phosphocellulose paper (Millipore, cat # MAPHNOB50) and was washed 3 times with 200 μL 0.5% H₃PO₄. Each phosphocellulase filter was transferred into a separate scintillation vial, 1 ml cytoscint fluid was added, and the sample counted on a Packard 1900TR liquid scintillation analyzer for 1 minute.

The compounds of Examples 1-11 were run with the recited assay and showed inhibitory activity versus ret-kinase shown in Table 2 below. Concentrations of Example 1 tested in the ret enzyme kinase assay to determine the IC₅₀ were 0, 0.5, 1, 5, 10, 50, 100, and 250 nM. The IC50 was determined from this data using linear regression analysis and Graphpad Prism 4 software.

| Example No. | Ret | c-src | VEFGR2 |
|---|---|---|---|
| 1 | *** | *** | ND |
| 2 | ** | ** | ND |
| 3 | ** | ** | *** |
| 4 | ** | *** | *** |
| 5 | ** | *** | *** |
| 6 | *** | | |
| 7 | ** | *** | ND |
| 8 | ** | *** | *** |
| 9 | ** | ** | ** |
| 10 | ** | ND | * |
| 11 | *** | *** | ** |

| | |
|---|---|
| Key: | |
| Symbol | range of activity, pIC50 values |
| *** | >8 |
| ** | 7–8 |
| * | 6–7 |
| ND | No data |

### Cellular Assays:

### MTT Assay:

The potency of the compound of the invention was tested for its ability to inhibit cell proliferation and cell viability. The metabolic conversion of 3-(4,5-dimethylthiazol-2-yl)-2,5-diphenyltetrazolium bromide (MTT, Sigma #M2128) to a reduced form is a commonly used measure of cellular viability.

TT cells, a medullary thyroid cancer cell line with constituitively activated ret, were maintained in 75 cm² tissue culture flasks until ready for use. The cells were grown and plated for the assay in F-12K Nutrient Mixture media containing 10% fetal bovine serum, 2 mM glutamine, 0.075% sodium bicarb, and penn/strep antibiotics. The cells were maintained at 37° C. in 10% CO₂, 90% humidified air. The cells were plated in 96-well tissue culture plates at 3-5×10³/well. 100 μL of cell suspension was added to each well of the 96-well plate except for the top row of the plate which contained no cells and served as a reference for the spectrophotometer.

The cells were incubated overnight in F-12K Nutrient Mixture media containing 10% fetal bovine serum, 2 mM glutamine, 0.075% sodium bicarb, and penn/strep antibiotics at 37° C. in 10% CO₂, 90% humidified air prior to dosing. The cells were dosed in sequential dilutions starting at 5 μM. 5 mM stock solutions of the compound were made in 100% dimethyl sulfoxide (DMSO). Stock solutions were diluted in F-12K Nutrient Mixture media containing 10% fetal bovine serum, 2 mM glutamine, 0.075% sodium bicarb, and penn/strep antibiotics. The final concentration of DMSO on the cells was kept below 0.3%. Serial dilutions were performed on the compound to prepare 2× concentrations of the compound for dosing. 100 μL of diluted compound was added to the 100 μL of media currently on the dish. For each concentration of compound, 6 replicate wells were prepared.

Cells were returned to the incubator and allowed to proliferate in the presence of compound for 168 hours before addition of MTT. MTT was prepared in phosphate buffered saline (Irvine Scientific #9240) at a concentration of 10 mg/mL. 10 μL per well of MTT solution was added to the 200 μL of media to yield a final concentration of 0.4 mg/mL and plates were returned to the incubator for 4 hours. After 4 hours incubation the media, compound and MTT mixture was aspirated from the plates and 100 μL of 100% DMSO was added to each well in addition to 25 μL of Sorenson's Buffer (0.1M glycine, 0.1M NaCl, pH 10.5). Quantitation of metabolic reduction of MTT in each plate was performed by reading optical density at 570 nm wavelength on a Bio-Rad 680 microplate reader. Concentrations of Example 1 examined were 0, 0.05, 0.1, 0.5, 1, 2.5 and 5μM. Growth inhibition curves and 50% inhibitory concentrations (IC₅₀) were determined using Microsoft Excel.

Examples were run with the recited assay and showed inhibitory activity versus TT cell proliferation. Representative data from examples are shown below.

| Example | IC50 |
|---|---|
| 5 | *** |
| 6 | *** |
| 8 | *** |
| 11 | *** |

| | |
|---|---|
| Key: | |
| Symbol | range of activity, IC50 values |
| *** | <1 uM |
| ** | 1–10 uM |
| * | 10–100 uM |
| ND | No data |

### Mechanistic Assay:

The potency of the compound of the invention was tested for its ability to inhibit constitutive ret kinase phosphorylation in cell culture.

TT cells, a medullary thyroid cancer cell line with constituitively activated ret, were maintained in 75 cm² tissue culture flasks until ready for use. The cells were grown and plated for the assay in F-12K Nutrient Mixture media containing 10% fetal bovine serum, 2 mM glutamine, 0.075% sodium bicarb, and penn/strep antibiotics. The cells were maintained at 37° C. in 10% CO₂, 90% humidified air. The cells were plated in 6-well tissue culture plates at 5×10⁵/well. The cells were incubated overnight in 4 ml F-12K Nutrient Mixture media containing 10% fetal bovine serum, 2 mM glutamine, 0.075% sodium bicarb, and penn/strep antibiotics at 37° C. in 10% CO₂, 90% humidified air prior to dosing. The compound of the invention was diluted in the same media to the final concentrations of 0, 0.1, 0.5, 1, 2.5, and 5 μM. The media in each well was then aspirated and the media containing the compound of the invention was added and incubated a further 24 hours at 37° C. in 10% CO₂, 90% humidified air.

Cells were harvested by aspirating the media and washing the cells with 2 ml phosphate buffered saline. Cells were then lysed directly by the addition of 125 μL 2× Laemmli (1% SDS, 10% glycerol, 100 mM dithiothreitol, 50 mM Tris, pH 6.8). All samples were sonicated briefly and boiled for 5 minutes, equivalent volumes were separated by SDS-PAGE (4-20% gradient), and electroblotted to nitrocellulose membranes. Blots were stained with amido black (0.5% in 5% acetic acid) and destained with water to confirm transfer and equal loading and then blocked in Tris buffered saline (0.8% NaCl, 2.7 mM KCl, 25 mM Tris, pH 7.4) with 0.05% Tween-20 (TBS-T) and 5% milk for 1 hour at 22° C. The blots were incubated in fresh blocking solution and probed for4 hours with a 1:1000 dilution of anti-phospho-RET (Santa Cruz Biotechnology) primary antibody. The blots were washed in 3 changes of TBS-T for 5 minutes each and then incubated with a 1:10,000 dilution of peroxidase-conjugated secondary antibody (Santa Cruz Biotechnology) in TBS-T for 1 hour at 22° C. Blots were again washed in 3 changes of TBS-T for 5 minutes each in TBS-T and

The phospho-ret signal on the Western blot was then quantified using ImageQuant software (Molecular Dynamics, Sunnyvale, Calif.) and the 50% inhibitory concentrations (IC₅₀) were determined using Microsoft Excel.

Representative data are shown below.

1. A method of treating a disease or condition associated with inappropriate ret kinase activity in a mammal, comprising administering to said mammal a compound of formula (Ia): 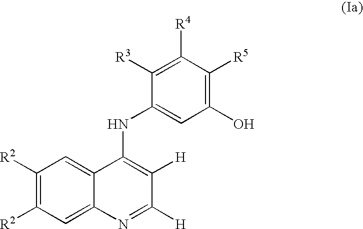
or a salt or solvate thereof, wherein
R¹ represents H, phenyl, a 5 or 6 membered monocyclic heteroaryl group, a bicyclic heteroaryl group, each of which phenyl, monocyclic or bicyclic heteroaryl group is optionally substituted by one or more substituents independently selected from
—C₁₋₃alkyl, —C₁₋₃hydroxyalkyl, —C₁₋₃haloalkyl, —C₁₋₃alkoxy, —C₁₋₃ haloalkoxy, —COH, —C₁₋₃alkyleneNR^{a}R^{b};
R^{a} and R^{b} are independently H, —C₁₋₃alkyl, —C₁₋₃alkyleneNR^{c}R^{d} or R^{a} and R^{b} , together with the N to which they are joined, form a 5 or 6 membered heterocyclic ring optionally containing a further heteroatom selected from O or N and optionally substituted by —C₁₋₃alkyl.
R^{c} and R^{d} are independently H, —C₁₋₃alkyl or
R^{c} and R^{d} together with the N to which they are joined form a 5 or 6 membered heterocyclic ring optionally containing a further heteroatom selected from O or N and optionally substituted by —C₁₋₃alkyl.
R² represents H, —SO₂C₁₋₃alkyl, —CONHC₁₋₃alkyl, a 6 membered monocyclic heteroaryl group.
R³, R⁴ and R⁵ independently represent H, halogen, —C₁₋₃alkyl with the proviso R¹ and R² are not both H.

2. The method as claimed in claim 1, wherein the compound of formula (Ia) is selected from:

| Ex. | R1 | R² | R³ | R⁴ | R⁵ |
|---|---|---|---|---|---|
| 1 | 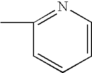 | H | H | H | H |
| 2 | 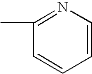 | H | H | CH₃ | H |
| 3 | 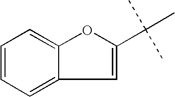 | H | H | H | Br |
| 4 | 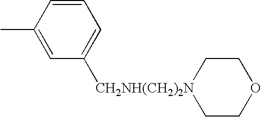 | H | F | H | Cl |
| 5 | 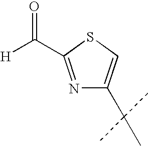 | H | CH₃ | H | H |
| 6 | H | —SO₂CH₃ | Cl | H | F |
| 7 | 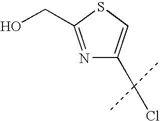 | H | Cl | H | F |
| 8 | 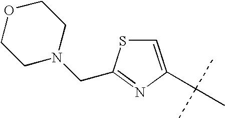 | —SO₂CH₃ | CH₃ | H | H |
| 9 | H | 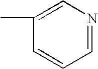 | CH₃ | H | H |
| 10 | H | 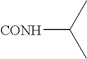 | CH₃ | H | H |
| 11 | H | —SO₂CH₃ | CH₃ | H | H |

3. A method as claimed in claim 1, wherein the disorder is thyroid cancer.

4. A method as claimed in claim 3, wherein the thyroid cancer is multiple endocrine neoplasia, familial thyroid carcinoma, papillary thyroid carcinoma, or Hirschsprungs Disease.

5. A method as claimed in claim 2, wherein the disorder is thyroid cancer.

6. A method as claimed in claim 5, wherein the thyroid cancer is multiple endocrine neoplasia, familial thyroid carcinoma, papillary thyroid carcinoma, or Hirschsprungs Disease.

7. A method of inhibiting ret kinase activity in a mammal, comprising administering to said mammal a compound of formula (Ia): 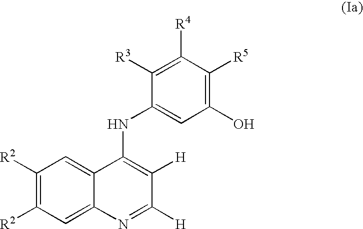
or a salt or solvate thereof, wherein
R¹ represents H, phenyl, a 5 or 6 membered monocyclic heteroaryl group, a bicyclic heteroaryl group, each of which phenyl, monocyclic or bicyclic heteroaryl group is optionally substituted by one or more substituents independently selected from
—C₁₋₃alkyl, —C₁₋₃hydroxyalkyl, —C₁₋₃haloalkyl, —C₁₋₃alkoxy, —C₁₋₃ haloalkoxy, —COH, —C₁₋₃alkyleneNR^{a}R^{b};
R^{a} and R^{b} are independently H, —C₁₋₃alkyl, —C₁₋₃alkyleneNR^{c}R^{d} or R^{a} and R^{b} , together with the N to which they are joined, form a 5 or 6 membered heterocyclic ring optionally containing a further heteroatom selected from O or N and optionally substituted by —C₁₋₃alkyl.
R^{c} and R^{d} are independently H, —C₁₋₃alkyl or
R^{c} and R^{d} together with the N to which they are joined form a 5 or 6 membered heterocyclic ring optionally containing a further heteroatom selected from O or N and optionally substituted by —C₁₋₃alkyl.
R² represents H, —SO₂C₁₋₃alkyl, —CONHC₁₋₃alkyl, a 6 membered monocyclic heteroaryl group.
R³, R⁴ and R⁵ independently represent H, halogen, —C₁₋₃alkyl with the proviso R¹ and R² are not both H.

8. The method as claimed in claim 7, wherein the compound of formula (Ia) is selected from:

| Ex. | R1 | R² | R³ | R⁴ | R⁵ |
|---|---|---|---|---|---|
| 1 | 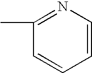 | H | H | H | H |
| 2 | 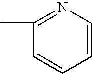 | H | H | CH₃ | H |
| 3 | 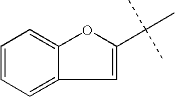 | H | H | H | Br |
| 4 | 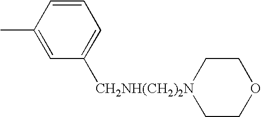 | H | F | H | Cl |
| 5 | 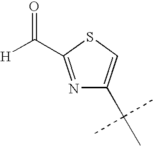 | H | CH₃ | H | H |
| 6 | H | —SO₂CH₃ | Cl | H | F |
| 7 | 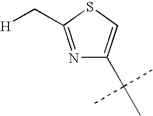 | H | Cl | H | F |
| 8 | 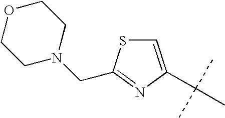 | —SO₂CH₃ | CH₃ | H | H |
| 9 | H | 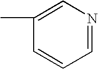 | CH₃ | H | H |
| 10 | H | 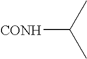 | CH₃ | H | H |
| 11 | H | —SO₂CH₃ | CH₃ | H | H |

9-12. (canceled)
